SEQUENCE LISTING (45 sequences)

SEQ ID NO: 1 - DNA, 20 bp, synthetic construct
  source 1..20: mol_type = other DNA
ttgatggcaacaatctccac

SEQ ID NO: 2 - DNA, 20 bp, synthetic construct
  source 1..20: mol_type = other DNA
cgtcccgtagacaaaatggt

SEQ ID NO: 3 - DNA, 23 bp, synthetic construct
  source 1..23: mol_type = other DNA
cccctagcctatatctccctttt

SEQ ID NO: 4 - DNA, 21 bp, synthetic construct
  source 1..21: mol_type = other DNA
taccatcgtggggataatggc

SEQ ID NO: 5 - DNA, 19 bp, synthetic construct
  source 1..19: mol_type = other DNA
tcagcctcctagctgttgg

SEQ ID NO: 6 - DNA, 22 bp, synthetic construct
  source 1..22: mol_type = other DNA
gtcaactctacactggttcctg

SEQ ID NO: 7 - DNA, 19 bp, synthetic construct
  source 1..19: mol_type = other DNA
ccgttgacccgctttctgt

SEQ ID NO: 8 - DNA, 21 bp, synthetic construct
  source 1..21: mol_type = other DNA
ttcggcgtccattttctttgg

SEQ ID NO: 9 - DNA, 22 bp, synthetic construct
  source 1..22: mol_type = other DNA
ctagctgtcactcaagggaaga

SEQ ID NO: 10 - DNA, 20 bp, synthetic construct
  source 1..20: mol_type = other DNA
cgaaggcgaacctcctctaa

SEQ ID NO: 11 - DNA, 21 bp, synthetic construct
  source 1..21: mol_type = other DNA
caggtgtgcttctgccaagat

SEQ ID NO: 12 - DNA, 21 bp, synthetic construct
  source 1..21: mol_type = other DNA
ggtaggtatccgtcatggtct

SEQ ID NO: 13 - DNA, 23 bp, synthetic construct
  source 1..23: mol_type = other DNA
gtcctcagaagctaaccatctcc

SEQ ID NO: 14 - DNA, 22 bp, synthetic construct
  source 1..22: mol_type = other DNA
ccagagcctatgactccatgtc

SEQ ID NO: 15 - DNA, 19 bp, synthetic construct
  source 1..19: mol_type = other DNA
gcaggcccagttatggctc

SEQ ID NO: 16 - DNA, 21 bp, synthetic construct
  source 1..21: mol_type = other DNA
caggctgaatgtaccctggtc

SEQ ID NO: 17 - DNA, 20 bp, synthetic construct
  source 1..20: mol_type = other DNA
gccaccgatgagatgatccc

SEQ ID NO: 18 - DNA, 20 bp, synthetic construct
  source 1..20: mol_type = other DNA
ttgatgtcggctaagtcgcc

SEQ ID NO: 19 - DNA, 21 bp, synthetic construct
  source 1..21: mol_type = other DNA
acagacttgagccggggatta

SEQ ID NO: 20 - DNA, 21 bp, synthetic construct
  source 1..21: mol_type = other DNA
tccattcaggagtagtgggag

SEQ ID NO: 21 - DNA, 21 bp, synthetic construct
  source 1..21: mol_type = other DNA
acccagagggacttatggaac

SEQ ID NO: 22 - DNA, 22 bp, synthetic construct
  source 1..22: mol_type = other DNA
gcagaatcttctagccctttgc

SEQ ID NO: 23 - DNA, 20 bp, synthetic construct
  source 1..20: mol_type = other DNA
ccctcgtggaggtaaagtgc

SEQ ID NO: 24 - DNA, 21 bp, synthetic construct
  source 1..21: mol_type = other DNA
ccttcccgatagacgacactc

SEQ ID NO: 25 - DNA, 21 bp, synthetic construct
  source 1..21: mol_type = other DNA
ctggcttctactccctgacct

SEQ ID NO: 26 - DNA, 21 bp, synthetic construct
  source 1..21: mol_type = other DNA
accagaacctagcatcaagga

SEQ ID NO: 27 - DNA, 21 bp, synthetic construct
  source 1..21: mol_type = other DNA
ccctgggaaaacactcacaca

SEQ ID NO: 28 - DNA, 22 bp, synthetic construct
  source 1..22: mol_type = other DNA
gcacaactcaatggtactgtcg

SEQ ID NO: 29 - DNA, 19 bp, synthetic construct
  source 1..19: mol_type = other DNA
gcggggacttctcgctatg

SEQ ID NO: 30 - DNA, 20 bp, synthetic construct
  source 1..20: mol_type = other DNA
ggctctgagagatcctgggg

SEQ ID NO: 31 - DNA, 21 bp, synthetic construct
  source 1..21: mol_type = other DNA
gccctgcactctcctgttttt

SEQ ID NO: 32 - DNA, 19 bp, synthetic construct
  source 1..19: mol_type = other DNA
ggttgccgcacagacttca

SEQ ID NO: 33 - DNA, 22 bp, synthetic construct
  source 1..22: mol_type = other DNA
ccaggatggttcttagactccc

SEQ ID NO: 34 - DNA, 21 bp, synthetic construct
  source 1..21: mol_type = other DNA
tttagcacgaagctctccgat

SEQ ID NO: 35 - DNA, 25 bp, synthetic construct
  source 1..25: mol_type = other DNA
agtcaacggatttggtcgtattggg

SEQ ID NO: 36 - DNA, 21 bp, synthetic construct
  source 1..21: mol_type = other DNA
acgtactcagcgccagcatcg

SEQ ID NO: 37 - DNA, 19 bp, synthetic construct
  source 1..19: mol_type = other DNA
ctcgctcactggtgaatca

SEQ ID NO: 38 - DNA, 19 bp, synthetic construct
  source 1..19: mol_type = other DNA
gattcgaaatggtgagaaa

SEQ ID NO: 39 - DNA, 19 bp, synthetic construct
  source 1..19: mol_type = other DNA
catccaagctgctacagaa

SEQ ID NO: 40 - DNA, 21 bp, synthetic construct
  source 1..21: mol_type = other DNA
ccaagtgctgccgtcattttc

SEQ ID NO: 41 - DNA, 22 bp, synthetic construct
  source 1..22: mol_type = other DNA
caatcacgctacctcctctttt

SEQ ID NO: 42 - DNA, 21 bp, synthetic construct
  source 1..21: mol_type = other DNA
tctcgaccctacatggcctta

SEQ ID NO: 43 - DNA, 19 bp, synthetic construct
  source 1..19: mol_type = other DNA
gccacggcacagtcattga

SEQ ID NO: 44 - DNA, 22 bp, synthetic construct
  source 1..22: mol_type = other DNA
ccaacccacctaacatttccag

SEQ ID NO: 45 - DNA, 20 bp, synthetic construct
  source 1..20: mol_type = other DNA
ggctggacgtgactcctaag

(19) United States
(12) Patent Application Publication
(10) Pub. No.: US 2026/0315555 A1
(43) Pub. Date: Oct. 15, 2026

(54) ONCOLYTIC VIRUS-INFECTED IMMUNE CELLS AND METHODS OF USE

(71) Applicant: Wake Forest University, Winston-Salem, NC (US)

(72) Inventors: Yong Lu, Winston-Salem, NC (US); Grant McFadden, Tempe, AZ (US)

(73) Assignee: Wake Forest University, Winston-Salem, NC (US)

(21) Appl. No.: 19/105,443

(22) PCT Filed: Aug. 23, 2023
(86) PCT No.: PCT/US2023/072719

§ 371 Date: Feb. 21, 2025

Related U.S. Application Data
(60) Provisional application No. 63/373,362, filed on Aug. 24, 2022.

(51) Int. Cl. A61K 35/76; A61K 35/17; A61P 35/00; C12N 5/0783

(52) U.S. Cl. CPC A61K 35/76; A61K 35/17; A61P 35/00; C12N 5/0636; C12N 2710/24032

(57) ABSTRACT

This disclosure is based, at least in part, on an unexpected discovery that oncolytic virus (e.g, myxoma virus)-infected tumor-specific T cells, expressing chimeric-antigen-receptor (CAR) or T-cell-receptor (TCR), are capable of inducing tumor cell autosis (in addition to T cell-induced apoptosis and pyroptosis) and/or overcoming primary and acquired resistance. The disclosed oncolytic virus (e.g, myxoma virus)-infected tumor-specific T cells represent a novel T-cell-cytotoxic machinery, and the unexpected synergy between T cells and oncolytic virus bolsters solid tumor cell autosis that reinforces tumor clearance.

### CROSS REFERENCE TO RELATED APPLICATION

This application claims priority to U.S. Provisional Application No. 63/373,362 filed on Aug. 24, 2022. The content of the application is incorporated herein by reference in its entirety.

### REFERENCE TO AN ELECTRONIC SEQUENCE LISTING

The contents of the electronic sequence listing (171567.00052_SeqListing.xml; Size: 39,600 bytes; and Date of Creation: Aug. 23, 2023) is herein incorporated by reference in its entirety.

### FIELD OF THE INVENTION

This invention relates to oncolytic virus-infected immune cells and methods of use thereof.

### BACKGROUND OF THE INVENTION

Adoptively transferred T cells engineered to express chimeric-antigen-receptor (CAR) have shown some success in eliminating hematopoietic cancers, but have so far been limited in their efficacy against solid tumors, which account for most cancer deaths. Lack of primary response (initial tumor regression) is most likely multifactorial and includes limited homing to and penetration of tumors, T cell exhaustion, limited persistence, and an immunosuppressive tumor microenvironment. Among those, interpatient and intratumoral heterogeneity of the tumor antigen may be one of the most serious impediments to efficacy against solid tumors and often lead to acquired resistance (recurrence after initial regression). Hence, it is urgent to develop novel strategies to overcome both primary and acquired resistance in solid tumors.

Oncolytic virotherapy is an emerging therapeutic modality for the treatment of cancer, but unfortunately, systemic delivery of oncolytic virus using standard intravenous infusion has thus far not achieved sufficient enrichment of virus in metastatic tumor beds. Although impressive responses can be obtained in the oncolytic virus-injected tumors, these approaches only induce limited or moderate responses to virus non-injectable metastasis tumors, which represents one major limitation for local virus administration.

Despite tremendous efforts, clinical trials of adoptive cell therapy (ACT) have not produced desired therapeutic responses in solid tumors. This may be in part because classic tumor cell apoptosis and pyroptosis induced by tumor-specific T cells with the formation of an immunological synapse are insufficient to trigger robust therapeutic responses in solid tumors.

Thus, there remains a pressing need for novel agents and methods of use thereof for treating or preventing cancer.

### SUMMARY OF THE INVENTION

This disclosure addresses the need mentioned above in a number of aspects. In one aspect, this disclosure provides a novel immune cell infected with an oncolytic virus (e.g., myxoma virus). The disclosed immune cells are capable of inducing tumor cell autosis and/or overcoming primary and acquired resistance.

In some embodiments, the oncolytic virus comprises a myxoma virus. In some embodiments, the oncolytic virus comprises a reporter gene.

In some embodiments, the immune cell is a lymphocyte. In some embodiments, the immune cell is a T cell. In some embodiments, the immune cell is a tumor-infiltrating T cell or a cytotoxic T lymphocyte.

In some embodiments, the immune cell is a T cell expressing a T cell receptor (TCR) or a chimeric antigen receptor (CAR). In some embodiments, the TCR or CAR binds specifically to an antigen on a tumor selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin. In some embodiments, the tumor comprises a solid tumor.

In some embodiments, the immune cell is infected by spin transduction with the oncolytic virus. In some embodiments, the spin transduction is performed at about 1,000 rpm to about 2,500 rpm (e.g., about 1,800 rpm) for about 1 hour to about 5 hours (e.g., 2 hours). In some embodiments, the immune cell is infected in the presence of protamine at a concentration ranging from about 5 μg/ml to about 15 μg/ml.

In another aspect, this disclosure further provides a composition comprising a plurality of immune cells described herein. In some embodiments, the composition further comprises non-infected immune cells. In some embodiments, a ratio of the infected immune cells to the non-infected immune cells is between 1:0.1 and 1:20. In some embodiments, the ratio of the infected immune cells to the non-infected immune cells is about 1:9.

Also within the scope of this disclosure is a kit comprising the immune cell or the composition, as described above.

In yet another aspect, this disclosure additionally provides a method of preparing the immune cell. In some embodiments, the method comprises: (a) introducing into a plurality of immune cells a vector comprising a nucleic acid sequence encoding a TCR or CAR to obtain a plurality of modified immune cells; (b) infecting the modified immune cells with an oncolytic virus; and (c) optionally culturing the infected immune cells in a cell culture medium.

In some embodiments, the oncolytic virus comprises a myxoma virus.

In some embodiments, infection of the modified immune cells is performed by spin transduction with the oncolytic virus. In some embodiments, the spin transduction is performed at about 1,000 rpm to about 2,500 rpm (e.g., about 1,800 rpm) for about 1 hour to about 5 hours (e.g., 2 hours). In some embodiments, the immune cell is infected in the presence of protamine at a concentration ranging from about 5 μg/ml to about 15 g/ml.

In some embodiments, the immune cells are autologous cells. In some embodiments, the immune cells are allogeneic cells.

In some embodiments, the modified immune cells are infected with the oncolytic virus at a multiplicity of infection (MOI) of about 0.1 to an MOI of about 10.

In another aspect, this disclosure also provides a method of treating cancer in a subject. In some embodiments, the method comprises administering to the subject an effective amount of the immune cells or the composition, as described above.

In another aspect, this disclosure further provides a method of treating cancer in a subject. In some embodiments, the method comprises: (a) introducing into a plurality of immune cells a vector comprising a nucleic acid sequence encoding a TCR or CAR to obtain a plurality of modified immune cells; (b) infecting the modified immune cells with an oncolytic virus; (c) optionally culturing infected immune cells in a cell culture medium; and (d) administering to the subject a composition comprising an effective amount of the infected immune cells.

In some embodiments, the immune cells are capable of inducing autosis of the cancer.

In some embodiments, the method comprises culturing the infected immune cells in a cell culture medium for a period of between 1 and 14 days. In some embodiments, the method comprises culturing the infected immune cells in the cell culture medium for a period of 7 days.

In some embodiments, the oncolytic virus comprises a myxoma virus.

In some embodiments, the composition further comprises non-infected immune cells. In some embodiments, a ratio of the infected immune cells to the non-infected immune cells is between 1:0.1 and 1:20. In some embodiments, the ratio of the infected immune cells to the non-infected immune cells is about 1:9.

In some embodiments, the immune cell is a T cell. In some embodiments, the immune cell is a tumor-infiltrating T cell or a cytotoxic T lymphocyte. In some embodiments, the immune cell is a T cell expressing a T cell receptor (TCR) or a chimeric antigen receptor (CAR). In some embodiments, the TCR or the CAR binds specifically to a tumor antigen selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin.

In some embodiments, the subject is human.

In some embodiments, the immune cells or the composition is administered by intravascular, subcutaneous, intraperitoneal, or intratumor injection.

In some embodiments, the cancer comprises a solid tumor or a hematologic malignancy. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the method further comprises administering to the patient an additional agent or therapy. In some embodiments, the additional agent comprises an anti-tumor or anti-cancer agent. In some embodiments, the additional agent or therapy is administered before, after, or concomitantly with administration of the composition.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features is not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, 1E, 1F, 1G, and 1H show delivery of MYXV by CAR-T cells.

FIG. 1A shows a protocol to prepare CAR-T^{MYXV} cells. On day 8, MYXV was added to CAR-T cells in the presence of 10 μg/ml protamine, and the culture was centrifuged at 1,800 rpm for 2 hrs for MYXV spin infection. FIG. 1B shows that the infection rate was detected 48 hrs after adding MYXV by FACS. FIG. 1C shows the yield of viable uninfected (PBS) and CAR-T^{MYXV} cells (n=3 donors/group). Data are mean±SD. **P<0.01, MYXV (Protamine/Spin) compared with others, two-way ANOVA with posthoc Holm-Sidak test. FIG. 1D shows that % of live CAR-T^{MYXV} cells were measured by trypan blue exclusion test (n=3). Data are mean±SD. *P<0.05, two-way ANOVA with posthoc Holm-Sidak test. FIG. 1E shows that MYXV-tdTomato (3×10⁴ FFUs), MSLN CAR-T^{MYXV-tdTomato} (1×10²), or CD19 CAR-T^{MYXV-tdTomato} (1×10²) was added to SKOV3 cells (1×10⁴). Representative images (scale bar, 15 μm) and % of tdTomato⁺ SKOV3 cells are shown (24 hrs; n=3-5/group). Data are mean±SD. ***P<0.001, MSLN CAR-T^{MYXV} compared with any other groups, one-way ANOVA with Tukey test. FIGS. 1F-H show treatment schema (FIG. 1F). Indicated treatments were given when tumors reached ~7×6 mm on day 35. In groups 1 and 3, PBS and MYXV-Luc⁺, respectively, were i.t. injected only into tumors on the left flanks (L. i.t.), and tumors on the right flanks did not receive i.t. injection. In group 2, MYXV-Luc⁺ and MSLN CAR-T cells were i.v. injected. In groups 3 and 4, MSLN CAR-T and MSLN CAR-T^{MYXV-Luc+} cells were i.v. injected, respectively. Bioluminescence was measured on day 42. Representative images (FIG. 1G) and summarized data are shown (FIG. 1H) (n=5/group). Data are mean±SD. ***P<0.001, group 3 (Left) compared with groups 1 & 2 (Left); **P<0.01, group 4 (Left) compared with groups 1 & 2 (Left); **P<0.01, group 4 (Right) compared with any other groups (Right), two-way ANOVA with posthoc Holm-Sidak test. Pooled results represent 2 independent experiments (FIGS. 1E and 1H).

FIGS. 2A, 2B, 2C, 2D, 2E, 2F, and 2G show that tumor-specific T^{10% MYXV} display strong antitumor efficacy. FIGS. 2A-B show schema of in vitro cytolytic assays. FIG. 2A shows that MSLN CAR-T or MSLN CAR-T^{10% MYXV} cells (2×10³) were cocultured with SKOV3 cells (2×10⁴). MART-1 T or MART-1 T^{10% MYXV} cells (2×10³) were cocultured with Mel-264 cells (2×10⁴). CD19 CAR-T or CD19 CAR-T^{10% MYXV} cells (2×10³) were cocultured with Raji cells (2×10⁴). Cytotoxic assays were performed every 8 hrs. FIG. 2B shows representative images (72 hrs) and summarized tumor-killing activity (n=3/time point). Data are mean±SD. ***P<0.001, SKOV3; **P<0.01, Mel-264; ***P<0.001, Raji, two-way ANOVA with posthoc Holm-Sidak test. FIGS. 2C-G show treatment schema. NSG mice bearing 40-day established SKOV3 tumors were treated when tumors reached ~9×8 mm on day 40 (FIGS. 2C-E); NSG mice bearing 10-day established Mel-264 tumors were treated when tumors reached ~7×6 mm on day 10 (FIGS. 2C and 2F-G). In groups 1, 2, 3, and 4, PBS or MYXV was i.t. injected only into tumors on the left flanks (L. i.t.), and tumors on the right flanks did not receive i.t. injection. In groups 3 and 4, MSLN CAR-T or MART-1 T cells were i.v. injected. In group 5, MART-1 T^{10% MYXV} or MSLN CAR-T^{10% MYXV} cells were i.v. injected. Tumor responses to ACT with MSLN CAR-T^{10% MYXV} (FIG. 2D) and MART-1 T^{10% MYXV} cells (FIG. 2F) are shown (n=5/group). Data are mean #SD. ***P<0.001, MSLN CAR-T^{10% MYXV} compared with any other groups (FIG. 2D); **P<0.01, MART-1 T^{10% MYXV} compared with any other groups (FIG. 2F), two-way ANOVA with posthoc Holm-Sidak test. FIGS. 2E and 2G show survival curves from 2 independent studies (n=10-12/group). ***P<0.001, compared with any other groups. Survival analysis was conducted by log-rank test.

FIGS. 3A, 3B, 3C, 3D, 3E, 3F, 3G, 3H, 3I, 3J, and 3K show that CAR-T^{10% MYXV} cells induce tumor cell autosis. FIG. 3A shows that MSLN CAR-T or MSLN CAR-T^{10% MYXV} cells (2×10³) were cocultured with SKOV3 cells (2×10⁴). Various cell death types after 72 hrs coculture are shown (indicated with arrows), including apoptosis (blue), pyroptosis (swelling with bubbles), and special cell death (autosis) with a strong attachment to culture plate. FIG. 3B shows that SKOV3 cells were seeded into the upper (2×10⁵) and lower (2×10⁴) Transwell chambers. MSLN CAR-T or MSLN CAR-T^{10% MYXV} cells (2×10⁴) were only added into upper chambers. Representative images for morphology of SKOV3 cells in the lower chamber are shown (72 hrs). FIG. 3C shows that SKOV3 cells were pretreated with BYK204165 (parthanatos; PARP-1 inhibitor, 5 μM), Y-27632 (entotic cell death; ROCK inhibitor, 1 μM), Lactoferrin (netotic cell death; NETs inhibitor, 2.8 μM) or CA-074Me (lysosome-dependent cell death; CTSB inhibitor, 5 nM) for 24 hrs. MYXV (6×10⁴ FFUs) or MSLN CAR-T^{10% MYXV} cells (2×10³) were then added to pretreated SKOV3 cells. % of killing was determined by an in vitro cytotoxicity assay (72 hrs, n=5). FIG. 3D shows that control (Ctrl) or ATP1A1 siRNA was transfected into SKOV3 cells 72 hrs before further treatments. MYXV (6×10⁴ FFUs), MSLN CAR-T, or MSLN CAR-T^{10% MYXV} cells (2×10³) were seeded to SKOV3 cells. % of killing was determined by an in vitro cytotoxicity assay (72 hrs, n=5). Data are mean±SD. *P<0.05, **P<0.01, two-way ANOVA with posthoc Holm-Sidak test. FIGS. 3E-I show that MYXV (3×10⁶ FFUs), MSLN CAR-T, or MSLN CAR-T^{10% MYXV} cells (1×10⁵) were cocultured with SKOV3 cells (1×10⁶) for 24 hrs. Tat-Beclin1 was resuspended (10 μM) and coincubated with SKOV3 cells for 1.5 hrs. FIG. 3E shows that lower chamber SKOV3 cells were treated as shown in FIG. 3B for 24 hrs. SKOV3 cell lysates (after T cell removal) or lower chamber SKOV3 cells were analyzed for protein expression levels by Western blot. FIGS. 3F-I show that total RNA was extracted from the SKOV3 cells for RNA-Seq (n=2). FIG. 3F shows hierarchical clustering of expression levels of 16,382 genes in treated SKOV3 cells. FIG. 3G shows correlation matrix of Pearson correlation values (PCV) calculated pairwise between all groups (top ~250 most changed GO term pathways). FIG. 3H shows GSEA results for GO term apoptosis pathway. FIG. 3I shows dotplot heatmap of enriched ion channel-related signaling analyzed by GSEA. FIGS. 3J-K show representative images of LC3 staining in SKOV3 cells treated with Tat-Beclin1 (10 μM), MYXV (6×10⁴ FFUs), MSLN CAR-T, or MSLN CAR-T^{10% MYXV} cells (2×10³) for 24 hrs. White arrows indicate representative LC3+ cells (scale bar, 10 μm) (FIG. 3J). Statistical analyses (FIG. 3K, n=5). Data are mean±SD. *P<0.05, ***P<0.001, one-way ANOVA with Tukey test. Pooled results represent 2 independent experiments.

FIGS. 4A, 4B, 4C, 4D, 4E, 4F, 4G, 4H, 4I, and 4J show that T cell-derived IFN-γ synergizes with MYXV-derived SKP-1/VPS34 signaling to induce tumor cell autosis. FIG. 4A shows that SKOV3 cells were treated as shown in FIG. 3E. SKOV3 cell lysates were analyzed for protein expression levels by Western blots. FIGS. 4B-D show that SKOV3 cells were transduced with Ctrl or human SKP-1 expression plasmids. FIG. 4B shows that SKOV3 cell lysates (24 hrs) were analyzed for protein expression levels by Western blots. FIG. 4C shows % of killing was determined by an in vitro cytotoxicity assay (72 hrs, n=5/group). Data are mean±SD. **P<0.01, two-way ANOVA with posthoc Holm-Sidak test. FIG. 4D shows that MSLN CAR-T or MSLN CAR-T^{10% MYXV} cells (2.5×10⁶) were transferred i.v. into NSG mice bearing SKOV3 tumors when tumors reached ~9×8 mm (1×10⁷ SKOV3-GFP cells or SKOV3-SKP-1 cells challenged s.c. 40 days before ACT). Survival curves from two independent studies are summarized (n=9-12/group). **P<0.01 compared with any other groups, survival analysis was conducted by log-rank test. FIG. 4E shows that in vitro cytotoxicity assay (72 hrs, n=5/group), SKOV3 cells (2×10⁴) were treated with MYXV (6×10⁴ FFUs) plus supernatant from SKOV3 and MSLN CAR-T cell coculture, and 10 μg/ml indicated mAbs (n=5/group). Data are mean±SD. **P<0.01, anti-IFN-g compared with IgG, one-way ANOVA with Tukey test. FIGS. 4F-G show that SKOV3 cells (2×10⁵) were seeded into the upper chambers. SKOV3 or gene-modified SKOV3 cells (2×10⁴) were seeded into lower chambers. MSLN CAR-T^{10% MYXV} cells (2×10⁴) were only added to upper chambers. % of killing was determined by an in vitro cytotoxicity assay (72 hrs, n=5). Data are mean±SD. **P<0.01, one-way ANOVA with Tukey test. FIG. 4H shows Western blot analysis of protein expression levels in SKOV3 cells with indicated treatments after T cell removal. (24 hrs, 10 ng/ml hIFN-g, 10 μg/ml anti-hIFN-g mAb). FIGS. 4I-J show that Ctrl vector or CA-AKT vector was transduced into SKOV3 cells. FIG. 4I shows that indicated treatment of MSLN CAR-T or MSLN CAR-T^{10% MYXV} cells (2×10³) was given for 24 hrs after transduction. % of killing was determined by in vitro cytotoxicity assays (72 hrs; n=5/group). Data are mean±SD. **P<0.01, two-way ANOVA with posthoc Holm-Sidak test. FIG. 4J shows that MYXV (6×10⁴ FFUs) or MSLN CAR-T^{10% MYXV} (2×10³) cells were given for 24 hrs after transduction. Western blot analysis of protein expression levels in SKOV3 cells with indicated treatments after T cell removal. Representative results from one of two (FIGS. 4E-G, 4I) repeated experiments are shown.

FIGS. 5A, 5B, 5C, 5D, 5E, 5F, and 5G show that murine tumor-specific T^{10% MYXV} ACT eradicates solid tumors with antigen heterogeneity. FIGS. 5A-C show that indicated treatments were given to B6 mice bearing s.c. established ID-8^{80% hMSLN} tumors on day 43 when tumors reached ~9×7 mm. FIG. 5A shows treatment schema. In groups 1, 2, and 3, PBS was i.t. injected only into tumors on the left flank (L. i.t.). In group 4, MYXV was i.t. injected only into tumors on the left flank (L. i.t.). In groups 3 and 4, MSLN CAR-T cells were i.v. injected. In group 5, MSLN CAR-T^{10% MYXV} cells were i.v. injected. FIG. 5B shows that tumor responses are shown (n=5/group). Data are mean±SD. **P<0.01, MSLN CAR-T^{10% MYXV} compared with any other groups, two-way ANOVA with posthoc Holm-Sidak test. FIG. 5C shows that survival curves from two independent studies are summarized (n=8-10/group). *P<0.05, compared with any other groups, survival analysis was conducted by log-rank test. FIGS. 5D-G show that B6 mice were inoculated s.c. with 1×10⁶ B16^{20% TRP-1-KO} cells only on the left flank and injected i.v. with 1×10⁵ B16^{20% TRP-1-KO} cells to induce lung metastatic tumors. Treatments were administrated on day 8 when s.c. tumors reached ~7×6 mm. FIG. 5D shows treatment schema. In groups 1, 2, and 3, PBS was i.t. injected only into tumors on the left flank (L. i.t.). In group 4, MYXV was i.t. injected only into tumors on the left flank (L. i.t.). In groups 3 and 4, TRP-1 T cells were i.v. injected. In group 5, TRP-1 T^{10% MYXV} cells were i.v. injected. FIG. 5E shows tumor responses (n=5/group). Data are mean±SD. ***P<0.001, TRP-1 T^{10% MYXV} compared with TRP-1 T group, two-way ANOVA with posthoc Holm-Sidak test. FIG. 5F shows survival curves from two independent studies (n=9-11/group). **P<0.01, compared with any other groups, survival analysis was conducted by log-rank test. FIG. 5G shows the counts of lung foci (n=5/group). Data are mean±SD. *P<0.05, TRP-1 T^{10% MYXV} compared with any other groups (Day 50), one-way ANOVA with Tukey test.

FIGS. 6A, 6B, 6C, 6D, 6E, 6F, 6G, and 6H show that ACT with T^{10% MYXV} induces a robust adaptive immunity to restrain ALVs. FIGS. 6A-F show that B16^{20% TRP-1-KO} cells were s.c. injected into both flanks of the mice. Treatments were administrated on day 8 when tumors reached ~7×6 mm. FIG. 6A shows treatment schema. In groups 1 and 2, PBS was i.t. injected only into tumors on the left flank (L. i.t.). In group 3, MYXV was i.t. injected only into tumors on the left flank (L. i.t.). In groups 2 and 3, TRP-1 T cells were i.v. injected. In group 4, TRP-1 T^{10% MYXV} cells were i.v. injected. FIGS. 6B-D show tumor tissues (~100 mg/mice, 12 days after ACT) that were harvested. Total RNA was extracted from the tumor tissues for RNA-seq (n=2). FIG. 6B shows hierarchical clustering of expression levels of 25,240 genes. Heatmap of enriched genes of GO terms ‘inflammatory response’ (FIG. 6C) and ‘adaptive immune response’ (FIG. 6D) and GSEA between indicated treatments are shown. (FIG. 6E) Log fold-change ratio versus mean average expression plot of differentially expressed genes suggesting antitumor immunity in tumors after TRP-1^{10% MYXV} ACT or CTX treatment. FIG. 6F shows IFN-γ ELISpot analysis of tumor-infiltrating CD4⁺ and CD8⁺ host T cells restimulated with irradiated B16^{TRP-1-KO} ALVs. Representative images and statistical results are shown (n=5/group). Data are mean±SD. ***P<0.001, TRP-1 T^{10% MYXV} compared with any other groups (CD8+ T cells); **P<0.01, TRP-1 T^{10% MYXV} compared with any other groups (CD4+ T cells), one-way ANOVA with Tukey test. Representative results from two repeated experiments are shown. FIG. 6G shows that TRP-1 T^{10% MYXV} cells (5×10⁶) were transferred i.v. into WT, CD4^{-/-}, CD8^{-/-} and CD3^{-/-} B6 mice when B16^{20% TRP-1-KO} tumors reached ~7×6 mm (similar to FIG. 6A). Survival curves from 2 independent studies are summarized (n=9-11/group). **P<0.01, compared with any other groups, survival analysis was conducted by log-rank test. FIG. 6H shows that WT and CD3^{-/-}B6 mice were s.c. inoculated with 1×10⁶ (80% B16+20% B16^{TRP-1-KO+Ctrl}) tumor cells. Some groups of mice were s.c. inoculated with 1×10⁶ (80% B16+20% B16^{TRP-1-KO+SKP-1}) tumor cells. TRP-1 T^{10% MYXV} cells (5×10⁶) were transferred i.v. into mice when tumors reached ~7×6 mm (similar to FIG. 6A). Survival curves from two independent studies are summarized (n=9-11/group). **P<0.01, compared with any other groups, survival analysis was conducted by log-rank test.

FIGS. 7A, 7B, 7C, 7D, 7E, 7F, 7G, 7H, 7I, 7J, 7K, and 7L show characteristics of MYXV-loaded T cells. FIG. 7A shows that γ-retrovirus encoding human MSLN BBZ CAR or CD19 BBZ CAR was transduced into the activated human T cells one day after activation. After expansion for 7 days, % of CD19 CAR⁺ and MSLN CAR⁺ T cells were detected by FACS. Representative results are shown. FIG. 7B shows that MYXV-tdTomato was added to CAR-T cells at MOI of 3:1 (MYXV:MSLN CAR-T) in the presence of 10 μg/ml protamine, and the culture was centrifuged at 1,800 rpm for 2 hrs for loading MYXV to CAR-T cells. The MYXV infection rate was detected by FACS 7 days after adding MYXV. FIG. 7C shows that the surface expression level of MSLN on SKOV3 cells was detected by FACS. FIG. 7D shows that MYXV-tdTomato (3×10⁴ FFUs), CD19 CAR-T^{MYXV-tdTomato}, or MSLN CAR-T^{MYXV-tdTomato} cells (1×10²) were added to Raji cells (1×10⁴). % of tdTomato⁺ Raji cells are shown (24 hrs; n=3-5/group). Data are mean±SD. **P<0.01, CD19 CAR-T^{MYXV} compared with any other groups, one-way ANOVA with Tukey test. FIGS. 7E-G show that MYXV-tdTomato titers (or replication) were determined using red foci formation on BSC40 cells. Titers for each sample were performed in triplicate. FIG. 7E shows that MSLN CAR-T^{MYXV-tdTomato} or MART-1 T^{MYXV-tdTomato} cells were cocultured with/without SKOV3 or Mel-264 cells, respectively. MSLN CAR-T^{MYXV-tdTomato} or MART-1 T^{MYXV-tdTomato} cells were isolated and lysed to determine MYXV replicates in MSLN CAR-T^{MYXV-tdTomato} and TRP-1 T^{MYXV-tdTomato} cells at indicated time points. FIG. 7F shows that MSLN CAR-T^{MYXV-tdTomato} or CD19 CAR-T^{MYXV-tdTomato} were cocultured with SKOV3 or Raji cells. Cell culture supernatant was collected after 72 hrs to quantify MYXV released from CAR-T^{MYXV-tdTomato} cells. FIG. 7G shows that CAR-T^{MYXV} (MSLN CAR-T^{MYXV}) and CAR-TΔZ^{MYXV} (MSLN CAR-T-BBΔZ^{MYXV}) were reactivated with MSLN beads or anti-CD3/28 beads, and CD19 beads were used as a negative control. Cell culture supernatant was collected 72 hrs after reactivation to determine MYXV released from CAR-T^{MYXV} or CAR-TΔZ^{MYXV} cells. Data are mean±SD. **P<0.01, one-way ANOVA with Tukey test. FIG. 7H shows that SKOV3 or Raji cells were seeded into the upper (2×10⁵) and lower (2×10⁴) Transwell chambers. MYXV-tdTomato (6×10⁵ FFUs), CD19 CAR-T^{MYXV-tdTomato}, or MSLN CAR-T^{MYXV-tdTomato} (2×10³) cells were only added to upper chambers. % of tdTomato⁺ SKOV3 or tdTomato⁺ Raji in the lower chamber was detected after 48 hrs.**P<0.01, MYXV compared with PBS (SKOV3), MSLN CAR-T^{MYXV} compared with PBS (SKOV3); MYXV compared with PBS (Raji), CD19 CAR-T^{MYXV} compared with PBS (Raji), one-way ANOVA with Tukey test. Representative results from one of two repeated experiments are shown. FIG. 7I shows that GFP⁺ MSLN CAR-T cells were seeded into the upper (2×10⁵) and lower (2×10⁴) Transwell chambers. MYXV-tdTomato (6×10⁴ FFUs), or GFP/tdTomato⁺ MSLN CAR-T^{MYXV} cells (2×10⁴) were only added to upper chambers. % of tdTomato⁺GFP⁺ MSLN CAR-T in the upper and lower chambers were detected after 48 hrs. Representative results from one of two repeated experiments are shown. FIGS. 7J-K show that NSG mice bearing 35-day established SKOV3 tumors. FIG. 7J shows that MSLN CAR-T^{MYXV-Luc+} cells were i.v. injected when tumors reached ~7×6 mm on day 35. Mice were euthanized on day 42. CD3⁺ T cells were isolated from both flanks of tumors. Bioluminescence of SKOV3 cells (T cell-depleted) and CD3⁺ T cells were detected. Pooled results represent 2 independent experiments. FIG. 7K shows that MSLN CAR-T^{MYXV-Luc+} or MSLN CAR-TΔZ^{MYXV-Luc+} cells were i.v. injected on day 35. Bioluminescence of mice was measured on day 42. Summarized data are shown (n=3/group). FIG. 7L shows that NSG mice bearing 35-day established MSLN-KO SKOV3 cells, indicated treatment schema was similar to FIG. 1F. Bioluminescence of mice was measured on day 42. Summarized data are shown (n=3/group).

FIGS. 8A, 8B, 8C, 8D, 8E, 8F, 8G, 8H, 8I, 8J, 8K, 8L, 8M, and 8N show that the optimal antitumor function requires CAR-T^{MYXV} tumor specificity. FIG. 8A shows that SKOV3 cells (1×10⁴) were treated with MYXV (3×10⁴ FFUs), MSLN CAR-T, or MSLN CAR-T^{MYXV} (1×10³). % of killing was determined by an in vitro cytotoxicity assay (72 hrs, n=3-5/group). FIG. 8B shows that SKOV3 cells (2×10⁴) were cocultured with 2×10³ MSLN CAR-T^{100% MYXV} (100% MSLN CAR-T^{MYXV}), MSLN CAR-T80% MYXV (80% MSLN CAR-T^{MYXV}+20% MSLN CAR-T), MSLN CAR-T60% MYXV (60% MSLN CAR-T^{MYXV}+40% MSLN CAR-T), MSLN CAR-T^{40% MYXV} (40% MSLN CAR-T^{MYXV}+60% MSLN CAR-T), MSLN CAR-T^{20% MYXV} (20% MSLN CAR-T^{MYXV}+80% MSLN CAR-T), MSLN CAR-T^{10% MYXV} (10% MSLN CAR-T^{MYXV}+90% MSLN CAR-T), MSLN CAR-T5% MYXV (5% MSLN CAR-T^{MYXV}+95% MSLN CAR-T), or MSLN CAR-T^{1% MYXV} (1% MSLN CAR-T^{MYXV}+99% MSLN CAR-T) cells. % of killing was determined by an in vitro cytotoxicity assay (72 hrs, n=5/group). Data are mean±SD. **P<0.01, compared with MSLN CAR-T^{100% MYXV}, one-way ANOVA with Tukey test. FIG. 8C shows that SKOV3 cells (2×10⁴) were treated with MSLN CAR-T cells (2×10³), MYXV (6×10⁴ FFUs), MSLN CAR-T^{MYXV} cells (2×10³), a mixture of 90% MSLN CAR-T (1.8×10³) and 10% MSLN CAR-T^{MYXV} cells (0.2×10³), CD19 CAR-T cells (2×10³), a mixture of 90% CD19 CAR-T (1.8×10³) and 10% CD19 CAR-T^{MYXV} cells (0.2×10³), a mixture of 90% MSLN CAR-T (1.8×10³) and 10% CD19 CAR-T^{MYXV} cells (0.2×10³), or a mixture of 90% CD19 CAR-T (1.8×10³) and 10% MSLN CAR-T^{MYXV} cells (0.2×10³). % of killing was determined by an in vitro cytotoxicity assay (72 hrs, n=5/group). Data are mean±SD. **P<0.01, 90% MSLN CAR-T+10% MSLN CAR-T^{MYXV} compared with any other groups, one-way ANOVA with Tukey test. FIG. 8D shows that hMSLN-expressing monocytes, hMSLN-expressing human umbilical vein endothelial cells (HUVECs), hMSLN-expressing epithelial (REPE-1) cells, and hMSLN-expressing stromal (HS-5) cells were generated by transduction with expression plasmid encoding hMSLN (monocytes^{MSLN}, HUVECs^{MSLN}, RWPE-1^{MSLN}, and HS-5^{MSLN}). SKOV3 cells, monocytes and monocytes^{MSLN}, HUVECs and HUVECs^{MSLN}, RWPE-1 and RWPE-1^{MSLN}, HS-5 and HS-5^{MSLN} (1×10⁴) cells were treated with MYXV-tdTomato (3×10⁴ FFUs) or MSLN CAR-T^{MYXV-tdTomato} cells (1×10³). % of tdTomato⁺ cells was detected after 48 hrs (n=3-5/group). FIG. 8E shows that monocytes and monocytes^{MSLN}, HUVECs and HUVECs^{MSLN}, RWPE-1 and RWPE-1^{MSLN}, HS-5 and HS-5^{MSLN} cells (1×10⁴) were treated with MYXV (3×10⁴ FFUs), MSLN CAR-T (1×10³), MSLN CAR-T^{10% MYXV} (1×10³), or MSLN CAR-T^{MYXV} cells (1×10³). % of killing was determined by an in vitro cytotoxicity assay (72 hrs, n=3-5/group). FIG. 8F shows that NSG mice bearing 40-day established SKOV3 tumors (1×10⁷), indicated treatments were given when tumors reached ~9×8 mm on day 40. In group 3, the combination of MSLN CAR-T cells (2.5×10⁶) and MYXV (2×10⁷ FFUs) were i.v. injected. Survival curves are shown (n=10-12/group). FIG. 8G shows that NSG mice bearing 40-day established SKOV3 tumors (1×10⁷). Indicated treatments were given when tumors reached ~9×8 mm on day 40. Survival curves are shown (n=10-12/group). FIGS. 8H-I show that mice were euthanized on day 5, day 10, or day 15 after treatments as shown in schema in FIG. 2C. FIG. 8H shows that CD3+ T cells were isolated from tumors and absolute CD3⁺ T cell # in about 300 mg tumor is shown (n=4-5). FIG. 8I shows that % of tdTomato⁺ MSLN CAR-T^{MYXV} cells (% of tdTomato⁺ CD3⁺ T cells). Pooled results represent 2 independent experiments. FIG. 8J shows that MYXV replication (tdTomato⁺ MYXV) in tumors was determined following indicated treatments. Treatment schema is shown in FIG. 2C. Mice were euthanized on day 5, day 10, day 15, or day 25 after treatments (n=4-5). Total tumor (CD3⁺ T cell-depleted) RNA was extracted, and tdTomato mRNA expression levels were analyzed by qPCR (n=3/group). **P<0.01, MYXV-tdTomato (i.t.)+MSLN CAR-T (Right) compared with any other groups, one-way ANOVA with Tukey test. FIG. 8K shows that mixed 90% CD19 CAR-T (4.5×10⁶) and 10% CD19 CAR-T^{MYXV} cells (0.5×10⁶), mixed 90% MSLN CAR-T (4.5×10⁶) and 10% CD19 CAR-T^{MYXV} cells (0.5×10⁶), 100% MSLN CAR-T^{MYXV} cells (5×10⁶), or mixed 90% MSLN CAR-T (4.5×10⁶) and 10% MSLN CAR-T^{MYXV} cells (0.5×10⁶) were transferred i.v. into NSG mice when SKOV3 tumors reached ~9×8 mm (1×10⁷ SKOV3 cells challenged s.c. 40 days before ACT). Survival curves are shown (n=9-12/group). ***P<0.001, compared with any other groups, survival analysis was conducted by log-rank test. FIG. 8L shows treatment schema that is shown in FIG. 2F. Mice were euthanized on day 8, day 16, or day 24 after treatments. CD3⁻ T cells were isolated from tumors, and absolute CD3⁺ T cell # in about 300 mg tumor is shown (n=4-5). Pooled results represent 2 independent experiments. FIG. 8M shows that MYXV replication (tdTomato⁺ MYXV) in tumors was determined following indicated treatments. Treatment schema is shown in FIG. 2C. Mice were euthanized on day 8, day 16, day 24, or day 32 after treatments (n=4-5). A total tumor (CD3⁺ T cell-depleted) RNA was extracted, and tdTomato mRNA expression levels were analyzed by qPCR (n=3/group). **P<0.01, MYXV-tdTomato (i.t.)+MART-1 T (Right) compared with any other groups, one-way ANOVA with Tukey test. FIG. 8N shows Raji tumor-bearing NSG mice (i.v. established) received indicated treatments on day 10. In group 3, MYXV and CD19 CAR-T cells were i.v. injected. In groups 2 and 4, CD19 CAR-T cells or CD19 CAR-T^{10% MYXV} cells were i.v. injected. Survival curves are shown (n=10-12/group). **P<0.01, compared with any other groups, survival analysis was conducted by log-rank test.

FIGS. 9A, 9B, 9C, 9D, 9E, 9F, 9G, 9H, 9I, 9J, 9K, 9L, 9M, and ON show the role of MYXV and MYXV-loaded CAR-T cells on tumor cells. FIG. 9A shows that SKOV3 cells were treated as shown in FIG. 3B. % of killing was determined by an in vitro cytotoxicity assay (72 hrs, n=5/group). ***P<0.001, MSLN CAR-T^{10% MYXV} compared with any other groups, one-way ANOVA with Tukey test. FIGS. 9B-C show that SKOV3, PANC1 (human pancreatic ductal cell line), U251 (human glioblastoma cell line), SK-BR-3 (human breast cancer cell line), and Mel-264 cells (2×10⁴) were treated with MYXV (6×10⁴ FFUs) at MOI of 3:1 for 72 hrs. Representative images for the morphology of tumor cells are shown. Cell death (autosis) with a strong attachment to the culture plate is indicated with an arrow. FIG. 9D shows that control (Ctrl) or ATP1A1 siRNA was transfected into SKOV3 cells for 72 hrs. Total RNA was extracted, and Atp1a1 mRNA expression levels were analyzed by qPCR (n=3/group). FIG. 9E shows that control (Ctrl) or ATP1A1 siRNA was transfected into Raji cells 72 hrs before further treatments. CD19 CAR-T or CD19 CAR-T^{10% MYXV} cells (2×10³) were seeded to Raji and ATP1A1-KD Raji cells (2×10⁴). After 72 hrs of coculture, % of killing was determined by an in vitro cytotoxicity assay (n=5). Data are mean±SD. **P<0.01, two-way ANOVA with posthoc Holm-Sidak test. FIGS. 9F-G show that SKOV3, Mel-264, Raji, SK-BR-3, PANC1, U251, and RPMI-8226 cells (2×10⁴) were treated with MYXV (6×10⁴ FFUs) at MOI of 3:1 for 72 hrs. FIG. 9F shows that Caspase 8 activity was measured by ELISA. **P<0.01, RPMI 8226+MYXV compared with RPMI-8226, one-way ANOVA with Tukey test. FIG. 9G shows representative images for the morphology of RPMI-8226 cells. Apoptotic cells are indicated with an arrow. FIG. 9H shows that tat-Beclin1 (10 μM) was coincubated with SKOV3 cells for 1.5 hrs. Representative images for the morphology of SKOV3 cells are shown. Cell death (autosis) with a strong attachment to the culture plate is indicated with an arrow. FIG. 9I shows that control (Ctrl) or ATP1A1 siRNA was transfected into SKOV3 cells 72 hrs before further treatments. Tat-Beclin1 (10 μM) was coincubated with SKOV3 cells for 1.5 hrs. % of killing was determined by an in vitro cytotoxicity assay (n=5). **P<0.01, Tat-Beclin1 treated ATP1A1-KD SKOV3 cells compared with Tat-Beclin1 treated Ctrl-KD SKOV3 cells, two-way ANOVA with posthoc Holm-Sidak test. FIG. 9J shows that SKOV3 cells were treated as shown in FIG. 3B. % of LC3+ cells were detected by immunofluorescence. Data are mean±SD. **P<0.01, MSLN CAR-T^{10% MYXV} compared with MSLN CAR-T, one-way ANOVA with Tukey test. FIGS. 9K-L show that GFP⁺ MSLN CAR-T cells, MSLN CAR-T^{MYXV}, or combined 90% GFP⁺ MSLN CAR-T cells and 10% MSLN CAR-T^{MYXV} cells were cocultured with SKOV3 cells. GFP⁺ MSLN CAR-T cells and MSLN CAR-T^{MYXV} were isolated from coculture after 72 hrs. FIG. 9K shows that isolated GFP⁺ MSLN CAR-T and MSLN CAR-T^{MYXV} cells were used for cytotoxicity assay against SKOV3 cells. % of killing was determined by an in vitro cytotoxicity assay (72 hrs; n=5/group). FIG. 9L shows absolute T cell # on 72 hrs (n=4-5). FIGS. 9M-N show that GFP⁺ MSLN CAR-T cells, MSLN CAR-T^{MYXV}, or combined 90% GFP⁺ MSLN CAR-T cells and 10% MSLN CAR-T^{MYXV} cells were cultured for 7 days. GFP⁺ MSLN CAR-T cells and MSLN CAR-T^{MYXV} were isolated from coculture after 7 days. FIG. 9M shows absolute T cell # on day 7 (n=4-5). (N) CD4⁺ T and CD8⁺ T cells were sorted from isolated GFP⁺ MSLN CAR-T and MSLN CAR-T^{MYXV} cells and extracted for total RNA. Sell, Il7r, Tcf7, Gzmb, Prf1, Lag3, Ctla4, and Pdcd1 mRNA expression levels were analyzed by qPCR (n=3/group).

FIGS. 10A, 10B, 10C, 10D, 10E, 10F, 10G, 10H, and 10I show effects of VPS34 and IFN-g. FIG. 10A shows that SKOV3 cells were pretreated with Ctrl (vehicle) or PIK-III (VPS34 inhibitor; 100 nM) for 12 hrs. MSLN CAR-T cells (2×10³), MYXV (6×10⁴ FFUs), or MSLN CAR-T^{10% MYXV} cells (2×10³) were then cocultured with pretreated SKOV3 cells. % of killing was determined by an in vitro cytotoxicity assay (72 hrs; n=5/group). Data are mean±SD. ***P<0.001, PIK-III (VPS34 inhibitor) compared with Ctrl group (with the treatment of MSLN CAR-T^{10% MYXV}), two-way ANOVA with posthoc Holm-Sidak test. FIG. 10B shows that control (Ctrl) or ATP1A1 siRNA was transfected into SKOV3 cells 72 hrs before further treatments. MSLN CAR-T cells (2×10³), MYXV (6×10⁴ FFUs), or MSLN CAR-T^{10% MYXV} cells (2×10³) were seeded to SKOV3 or ATP1A1-KD SKOV3 cells. After 72 hrs of coculture, % of killing was determined by an in vitro cytotoxicity assay (n=5). Data are mean±SD. ***P<0.001, two-way ANOVA with posthoc Holm-Sidak test. FIG. 10C shows that SKOV3 cells were seeded into the upper (2×10⁵) and lower (2×10⁴) Transwell chambers. MYXV (6×10⁵ FFUs) and culture supernatant (from coculture of SKOV3 and MSLN CAR-T cells) were only added to upper chambers. IgG or anti-IFN-g mAb (10 μg/ml) was added to the upper chambers. % of killing was determined by an in vitro cytotoxicity assay (n=5). Data are mean±SD. **P<0.01, one-way ANOVA with Tukey test. FIG. 10D shows that MYXV and culture supernatant (from coculture of SKOV3 and MSLN CAR-T cells), after filtration with 0.45 μm or 0.22 μm, were added into SKOV3 cells. % of killing was determined by an in vitro cytotoxicity assay (n=5). Data are mean±SD. FIG. 10E shows that SKOV3 cells (2×10⁴) were treated with MYXV (6×10⁴ FFUs), MYXV (6×10⁴ FFUs) plus hIFN-g (10 ng/ml), PIK-III (VPS34 inhibitor; 100 nM), or PIK-III (100 nM) plus hIFN-g (10 ng/ml). % of killing was determined by an in vitro cytotoxicity assay (72 hrs; n=5/group). Data are mean±SD. **P<0.01, one-way ANOVA with Tukey test. FIG. 10F shows that control (Ctrl) or ATP1A1 siRNA was transfected into SKOV3 cells 72 hrs before further treatments. MYXV (6×10⁴ FFUs) or MYXV (6×10⁴ FFUs) plus hIFN-g (10 ng/ml) were seeded to SKOV3 cells. After 72 hrs of coculture, % of killing was determined by an in vitro cytotoxicity assay (n=5). Data are mean±SD. ***P<0.001, two-way ANOVA with posthoc Holm-Sidak test. FIG. 10G shows that SKOV3 cells (2×10⁴) were treated with MSLN CAR-T cells (2×10³), MYXV (6×10⁴ FFUs), MSLN CAR-T^{MYXV} or MSLN CAR-T^{10% MYXV} cells (2×10³) for 72 hrs. Supernatants were analyzed by ELISA for hIFN-g concentrations (n=3/group). FIG. 10H shows that MYXV and culture supernatant (from coculture of SKOV3 and MSLN CAR-T cells), after filtration with 0.45 μm or 0.22 μm, were added into SKOV3 and gene-modified SKOV3 cells. % of killing was determined by an in vitro cytotoxicity assay (n=5). Data are mean±SD. FIG. 10I shows that MSLN CAR-T or MSLN CAR-T^{10% MYXV} cells (2.5×10⁶) were transferred i.v. into NSG mice bearing SKOV3 tumors when tumors reached ~9×8 mm (1×10⁷ SKOV3-Ctrl KO cells or 1×10⁷ SKOV3-IFNGR1-KO cells challenged s.c. 40 days before ACT). Survival curves from two independent studies are summarized (n=9-11/group). **P<0.01, compared with any other groups, survival analysis was conducted by log-rank test.

FIGS. 11A, 11B, 11C, 11D, 11E, 11F, and 11G show characterization of ID-8^{80% hMSLN} and B16^{20% TRP-1-KO} tumors. FIG. 11A shows that mice were treated as shown in FIG. 5A. Tumor tissues from the CTX- and MSLN CAR-T cell-treated mice were harvested at the endpoint and extracted for total RNA. ID-8 cells and ID-8^{80% hMSLN} cells were also extracted for total RNA and used as controls. hMSLN mRNA expression levels were analyzed by qPCR (n=3/group). Data are mean±SD. **P<0.01, MSLN CAR-T cells compared with CTX group, one-way ANOVA with Tukey test. FIG. 11B shows that mice were treated as shown in FIG. 5A. Tumor tissues from the CTX-, MSLN CAR-T cell- and MYXV (i.t.)+MSLN CAR-T cell-treated mice were harvested at the endpoint. Antigen expression levels were analyzed by IMC (n=3/group) and summarized data are shown (n=3/group). Data are mean±SD. ***P<0.001, CTX compared with any other groups, one-way ANOVA with Tukey test. FIG. 11C shows that MSLN CAR-T^{10% MYXV} cells were i.v. injected to B6 mice bearing s.c. established ID-8^{50% hMSLN} tumors (containing 50% WT ID-8 cells as ALVs) or ID-8^{80% hMSLN} tumors (containing 20% WT ID-8 cells as ALVs) on day 43 when tumors reached ~9×7 mm. Survival curves from two independent studies are summarized (n=8-10/group). **P<0.01, MSLN CAR-T^{10% MYXV} compared with MSLN CAR-T (ID-8^{50% hMSLN} tumors), survival analysis was conducted by log-rank test. FIG. 11D shows that MSLN CAR-T^{10% MYXV} cells were i.v. injected to B6 mice bearing s.c. established ID-8 tumors (100% WT ID-8 cells) or ID-8^{80% hMSLN} tumors on day 43 when tumors reached ~9×7 mm. Survival curves from two independent studies are summarized (n=8-10/group). FIG. 11E shows that B16^{20% TRP-1-KO} tumor (containing 20% B16 TRP-1-KO ALVs)-bearing mice were treated similar to FIG. 5D. Surviving mice from the TRP-1 T^{10% MYXV}-treated group were re-challenged with 3×10⁵ ALVs (B16^{TRP-1-KO}) on day 60. Survival curves from two independent studies are summarized (n=8-10/group). ***P<0.001, survival analysis was conducted by log-rank test. FIG. 11F shows that mice were treated as shown in FIG. 5D. Tumor tissues from the CTX- and TRP-1 T cell-treated mice were harvested at the endpoint and extracted for total RNA. B16^{TRP-1-KO} cells and B16^{20% TRP-1-KO} cells were also extracted for total RNA and used as controls. Tyrp1 (gene encoding TRP-1) mRNA expression levels were analyzed by qPCR (n=3/group). Data are mean±SD. **P<0.01, TRP-1 T cells compared with CTX group, one-way ANOVA with Tukey test. FIG. 11G shows that B6 mice were inoculated s.c with B16 or B16^{20% TRP-1-KO} cells (1×10⁶) without receiving any treatments. Survival curves are shown (n=10-12/group).

FIGS. 12A, 12B, 12C, 12D, and 12E show effects of host immune cells. FIG. 12A shows that mice were treated as shown in FIG. 5D. Tumor tissues from the CTX-, TRP-1 T cell- and MYXV (i.t.)+TRP-1 T cell-treated mice were harvested at the endpoint. Antigen expression levels were analyzed by IMC (n=3/group) and summarized data are shown (n=3/group). Data are mean±SD. **P<0.01, CTX compared with any other groups, one-way ANOVA with Tukey test.

FIG. 12B shows that mice were treated as shown in FIG. 6A. Tumor tissues from the CTX- and TRP-1 T^{10% MYXV} cell-treated mice were harvested at the endpoint and extracted for total RNA. Cd3, Cd8, Cd4, Cxcl10, Ccr7, Il12a, Il12b, Gzmb, Slcl1a1, Ifng, and Lef1 mRNA expression levels were analyzed by qPCR (n=3/group). Data are mean±SD. ***P<0.001, TRP-1 T^{10% MYXV} cell-treated group compared with CTX-treated group, one-way ANOVA with Tukey test. FIG. 12C shows that mice were treated as shown in FIG. 6A. Tumor tissues from the TRP-1 T, MYXV-Luc⁺ (L. i.t.)+TRP-1 (Left), and TRP-1 T^{10% MYXV-Luc+}-treated mice were harvested on day after treatments. MYXV infection was analyzed by IMC (n=3/group). FIG. 12D shows that TRP-1 T cells (5×10⁶) were transferred i.v. into WT, CD4^{-/-}, CD8^{-/-}, and CD3^{-/-} B6 mice when B16^{20% TRP-1-KO} tumors reached ~7×6 mm (1×10⁶ B16^{20% TRP-1-KO} challenged s.c. 8 days before ACT; adjuvant CTX was administered i.p. to mice one day before ACT). Survival curves are shown (n=9-11/group). FIG. 12E shows that WT and CD3^{-/-} B6 mice were s.c. inoculated with B16 tumor cells (1×10⁶). TRP-1 T^{10% MYXV} cells (5×10⁶) were transferred i.v. into mice when tumors reached ~7×6 mm on day 8. Adjuvant CTX was administered i.p. to mice one day before ACT. Survival curves are shown (n=9-11/group).

### DETAILED DESCRIPTION OF THE INVENTION

This disclosure is based, at least in part, on an unexpected discovery that oncolytic virus (e.g., myxoma virus)-infected tumor-specific T cells, expressing chimeric-antigen-receptor (CAR) or T-cell-receptor (TCR), are capable of inducing tumor cell autosis (in addition to T cell-induced apoptosis and pyroptosis) and/or overcoming primary and acquired resistance. The disclosed oncolytic virus (e.g., myxoma virus)-infected tumor-specific T cells represent a novel T-cell-cytotoxic machinery, and the unexpected synergy between T cells and oncolytic virus bolsters solid tumor cell autosis that reinforces tumor clearance.

Immune Cells Infected with Oncolytic Virus and Compositions Thereof
a. Oncolytic Virus-Infected Immune Cells

In one aspect, this disclosure provides a mammalian immune cell infected with an oncolytic virus.

In some embodiments, the immune cell comprises an immune effector cell. “Immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

In some embodiments, the immune cell is a lymphocyte. Lymphocytes are one subtype of white blood cells in the immune system. In some embodiments, lymphocytes may include tumor-infiltrating immune cells. Tumor-infiltrating immune cells consist of both mononuclear and polymorphonuclear immune cells (i.e., T cells, B cells, natural killer cells, macrophages, neutrophils, dendritic cells, mast cells, eosinophils, basophils, etc.) in variable proportions. In some embodiments, lymphocytes may include tumor-infiltrating lymphocytes (TILs). Tumor-infiltrating lymphocytes are white blood cells that have left the bloodstream and migrated towards a tumor. Tumor-infiltrating lymphocytes can often be found in the tumor stroma and within the tumor itself. In some embodiments, tumor-infiltrating lymphocytes are “young” T cells or minimally cultured T cells.

In some embodiments, lymphocytes may include peripheral blood lymphocytes (PBLs). In some embodiments, lymphocytes include T lymphocytes (T cells) and/or natural killer cells (NK cells).

In some embodiments, the lymphocytes may be autologous, allogeneic, syngeneic, or xenogeneic with respect to the subject. In some embodiments, the lymphocytes are autologous in order to reduce an immunoreactive response against the lymphocyte when reintroduced into the subject for immunotherapy treatment.

In some embodiments, the immune cell is a T cell. In some embodiments, the immune cell is a tumor-infiltrating T cell or a cytotoxic T lymphocyte.

In some embodiments, the immune cell is a T cell expressing a T cell receptor (TCR) or a chimeric antigen receptor (CAR).

As used herein, the term “T cell receptor” or “TCR” refers to a surface protein of a T cell that allows the T cell to recognize an antigen (e.g., tumor-associated antigen) and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an α chain and a β chain. Approximately 5% of T cells have TCRs made up of γ/δ chains. TCRs are membrane-anchored heterodimers that are found as part of a complex with a CD3 chain molecule. Each chain comprises two extracellular domains: a variable (V) region and a constant (C) region, the latter of which is membrane-proximal. The variable domains of α chains and β chains consist of three hypervariable regions that are also referred to as the complementarity determining regions (CDRs). The CDRs, in particular CDR3, are primarily responsible for contacting antigens and thus define the specificity of the TCR, although CDR1 of the α chain can interact with the N-terminal part of the antigen. CDR1 of the β chain interacts with the C-terminal part of the peptide. TCRs are also characterized by a series of highly conserved disulfide bonds that link the two chains.

The term “Chimeric Antigen Receptor” or alternatively a “CAR” refers to a chimeric antigen receptor (CAR). Chimeric antigen receptors (CARs) typically have an antigen-binding domain that is fused to an intracellular signaling domain which is capable of activating or stimulating an immune cell. The CAR's extracellular binding domain may be composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). The scFv may be fused to a transmembrane domain and then to an intracellular signaling domain. The CAR can be a first-generation, second generation or third-generation CAR. “First-generation” CARs include those that solely provide CD3z signals upon antigen binding. “Second-generation” CARs include those that provide both costimulation (e.g., CD28 or CD137) and activation (E\ddot{υ}3z). “Third-generation” CARs include those that provide multiple costimulation (e.g., CD28 and CD137) and activation (E\ddot{υ}3z). The CAR may specifically recognize a cancer antigen.

The terms “tumor-associated antigen (TAA),” “cancer antigen,” and “tumor antigen,” as used herein, refer to any molecule (e.g., protein, peptide, lipid, carbohydrate, etc.) solely or predominantly expressed or over-expressed by a tumor cell and/or a cancer cell, such that the antigen is associated with the tumor and/or the cancer. The TAA/cancer antigen can also be expressed by normal, non-tumor, or non-cancerous cells. However, in such a situation, the expression of the TAA/cancer antigen by normal, non-tumor, or non-cancerous cells is in some embodiments not as robust as the expression of the TAA/cancer antigen by tumor and/or cancer cells. Thus, in some embodiments, the tumor and/or cancer cells overexpress the TAA and/or express the TAA at a significantly higher level as compared to the expression of the TAA by normal, non-tumor, and/or non-cancerous cells. In some embodiments, the phosphopeptides are fragments of TAAs or TAAs themselves.

The TAA can be an antigen expressed by any cell of any cancer or tumor, including the cancers and tumors described herein. The TAA can be a TAA of only one type of cancer or tumor, such that the TAA is associated with or characteristic of only one type of cancer or tumor. Alternatively, the TAA can be characteristic of more than one type of cancer or tumor. For example, the TAA can be expressed by both breast and prostate cancer cells and not expressed at all by normal, non-tumor, or non-cancer cells.

Non-limiting examples of tumor-associated proteins from which tumor antigens (including neoantigens) can be identified include, e.g., 13HCG, 43-9F, 5T4, 791Tgp72, adipophilin, AIM-2, ALDH1A1, alpha-actinin-4, alpha-fetoprotein (“AFP”), ARTC1, B-RAF, BAGE-1, BCA225, BCLX (L), BCR-ABL fusion protein b3a2, beta-catenin, BING-4, brain glycogen phosphorylase, BTAA, c-met, CA-125, CA-15-3 (CA 27.29\BCAA), CA-19-9, CA-242, CA-50, CA-72-4, CALCA, CAM 17.1, CAM43, carcinoembryonic antigen (“CEA”), CASP-5, CASP-8, CD274, CD45, CD68\KP1, Cdc27, CDK12, CDK4, CDK 2A, CEA, CLPP, CO-029, COA-1, CPSF, CSNK1A1, CT-7, CT9/BRDT, CTAG1, CTAG2, CTp11, cyclin D1, Cyclin-A1, dek-can fusion protein, DK 1, E2A-PRL, EBNA, EF2, EFTUD2, Elongation factor 2, ENAH (hMena), Ep-CAM, EpCAM, EphA3, epithelial tumor antigen (“ETA”), Epstein Barr virus antigens, ETV6-AML1 fusion protein, EZH2, FGF5, FLT3-ITD, FN1, G250, G250/MN/CALX, Ga733 (EpCAM), GAGE-1,2,8, GAGE-3,4,5,6,7, GAS7, glypican-3, GnTV, gplOO, gplOO/Pmel 17, GPNMB, H-ras, H4-RET, HAUS3, Hepsin, HER-2/neu, HERV-K-MEL, HLA-A11, HLA-A2, HLA-DOB, HOM-MD-21, HOM-MD-397, Horn/Me 1-40, Horn/Mel-55, HPV E2, HPV E6, HPV E7, hsp70-2, HTgp-175, IDO1, IGF2B3, IGH-IGK, IL13Ralpha2, Intestinal carboxyl esterase, K-ras, Kallikrein 4, KIAAO205, KIF20A, KK-LC-1, KKLC1, KM-HN-1, KMHN1 also known as CCDC110, LAGE-1, LAGE-2, LB33/MUM-1, LDLR-fucosyltransferaseAS fusion protein, Lengsin, M-CSF, M344, MA-50, MAGE-1, MAGE-2, MAGE-3, MAGE-4, MAGE-5, MAGE-6, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, MAGE-B (MAGE-B 1-MAGE-B24), MAGE-C(MAGE-C1/CT7, CT10), MAGE-C1, MAGE-C2, MAGE-Xp2 (MAGE-B2), MAGE-Xp3 (MAGE-B3), MAGE-Xp4 (MAGE-B4), malic enzyme, mammaglobin-A, MAPE, MART-I, MART-2, MATN, MC1R, MCSP, mdm-2, ME1, Melan-A/MART-1, Meloe, MG7-Ag, Midkine, MMP-2, MMP-7, MOV 18, MUC1, MUC5AC, mucin, MUM-1, MUM-2, MUM-3, MYL-RAR, Myosin, Myosin class I, N-ras, N-raw, NA88-A, NAG, NBU70K, neo-PAP, NFYC, nm-23H1, NuMa, NY-BR-1, NY-CO-1, NY-CO-2, NY-ESOI, NY-ESO-I/LAGE-2, OA1, OGT, OS-9, P polypeptide, p15 (58), p16, p185erbB2, p180erbB-3, p53, PAP, PAX5, PBF, pm1-RARalpha fusion protein, polymorphic epithelial mucin (“PEM”), PPP1R3B, PRAME, PRDX5, PSA, PSCA, PSMA, PTPRK, RAB38/NY-MEL-1, RAGE-1, RBAF600, RCAS1, RGS5, R oC, RNF43, RU2AS, SAGE, SART-1, SART-3, SCP-1, SDCCAG16, secernin 1, SIRT2, SNRPD1, SOX10, Sp17, SPA17, SSX-1, SSX-2, SSX-4, SSX-5, STEAP1, survivin, SYT-SSX1 or -SSX2 fusion protein, TA-90 (Mac-2 binding protein\cyclophilin C-associated protein), TAAL6, TAG-1, TAG-2, TAG-72-4, TAGE, Telomerase, TERT, TGF-betaRII, TLP, TPBG, TPS TRAG-3, Triosephosphate isomerase, TRP-1, TRP-2, TRP-1/gp75, TRP-2, TRP2-INT2, TSP-180, TSP50, tyrosinase, tyrosinase (“TYR”), VEGF, WT1, XAGE-1b/GAGED2a, Kras, WT-1 antigen (in lymphoma and other solid tumors), ErbB receptors, Melan A (MARTI), gp 100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin (MUC-1) (in breast, pancreas, colon, and prostate cancers); prostate-specific antigen (PSA) (in prostate cancer); carcinoembryonic antigen (CEA) (in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-1/LAGE-2, NA-88, GnTV, TRP2-INT2. For example, antigenic peptides characteristic of tumors include those listed in Cancer Vaccines and Immunotherapy (2000) Eds Stern, Beverley and Carroll, Cambridge University Press, Cambridge, Cancer Immunology (2001), Kluwer Academic Publishers, The Netherlands, International Patent Application Publication No. WO 20000/020581 and U.S. Patent Application Publication No. 2010/0284965, and www.cancerimmunity.org/peptidedatabase/Tcellepitopes, which are each incorporated herein by reference in their entirety.

As used herein, the term “neoantigen” refers to a newly formed antigenic determinant that arises from a somatic mutation(s) and is recognized as “non-self.” A neoantigen can include a polypeptide sequence or a nucleotide sequence. A mutation can include a frameshift or non-frameshift indel, missense or nonsense substitution, splice site alteration (e.g., alternatively spliced transcripts), genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen (see, e.g., Liepe et al., Science; 354(6310):354-358 (2006), incorporated herein by reference in its entirety). A neoantigen can include a canonical antigen. A neoantigen can also include non-canonical antigen. Neoantigen can be tumor-specific.

In some embodiments, the TCR or CAR binds specifically to an antigen on a tumor selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin. In some embodiments, the tumor comprises a solid tumor.

As used herein, the phrase “specific binding” refers to binding between a TCR, CAR, or antigen-binding fragment thereof and an antigen and/or an epitope thereof (including but not limited to a peptide, optionally in complex with an MHC molecule) that is indicative of the presence of the antigen and/or the epitope thereof. As such, a TCR, CAR, or antigen-binding fragment thereof is said to “specifically” bind an antigen and/or an epitope thereof when the dissociation constant (Kd) is less than about 1 μM, less than about 100 nM, or less than about 10 nM. Interactions between a TCR, TCR-like molecule, or antigen-binding fragment thereof and an epitope can also be characterized by an affinity constant (Kₐ). In some embodiments, a Kₐ of less than about 10⁷/M is considered “high affinity.”

Oncolytic viruses may be engineered or naturally evolved viruses. In some embodiments, the oncolytic virus may be a replication-competent oncolytic rhabdovirus. Such oncolytic rhabdoviruses include, without limitation, Arajas virus, Chandipura virus, Cocal virus, Isfahan virus, Maraba virus, Piry virus, Vesicular stomatitis Alagoas virus, Vesicular stomatitis virus (VSV), BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Quango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island virus, Adelaide River virus, Berrimah virus, Kimberley virus, Rabies virus, or Bovine ephemeral fever virus.

In some embodiments, the oncolytic virus is a myxoma virus. Myxoma virus is a poxvirus in the genus Leporipoxvirus. Myxoma virus is a large virus with a double-stranded DNA genome of 163 kb, which replicates in the cytoplasm of infected cells (B. N. Fields, D. M. Knipe, P. M. Howley, Eds., Virology Lippincott Raven Press, New York, 2nd ed., 1996). Myxoma virus is known to encode a variety of cell-associated and secreted proteins that have been implicated in down-regulation of the host's immune and inflammatory responses and inhibition of apoptosis of virus-infected cells. Myxoma virus can be taken up by all human somatic cells.

Myxoma virus may be any virus that belongs to the Leporipoxvirus genus of pox viruses that is replication-competent. The Myxoma virus may be a wild-type strain of Myxoma virus or it may be a genetically modified strain of Myxoma virus.

In some embodiments, myxoma virus genome may be readily modified to express one or more transgenes using standard molecular biology techniques known to a skilled person, and described, for example, in Sambrook et al. ((2001) Molecular Cloning: a Laboratory Manual, 3rd ed., Cold Spring Harbour Laboratory Press). A skilled person will be able to readily determine which portions of the Myxoma viral genome can be deleted such that the virus is still capable of productive infection. For example, non-essential regions of the viral genome that can be deleted can be deduced from comparing the published viral genome sequence with the genomes of other well-characterized viruses (see, for example, C. Cameron, S. Hota-Mitchell, L. Chen, J. Barrett, J.-X. Cao, C. Macaulay, D. Wilier, D. Evans, and G. McFadden, Virology (1999) 264:298-318)).

In some embodiments, the transgenes may include a reporter gene, such as luciferase, GFP, etc.

In yet another aspect, this disclosure additionally provides a method of preparing the immune cell. In some embodiments, the method comprises: (a) introducing into a plurality of immune cells a vector comprising a nucleic acid sequence encoding a TCR or CAR to obtain a plurality of modified immune cells; (b) infecting the modified immune cells with an oncolytic virus; and (c) optionally culturing the infected immune cells in a cell culture medium.

In some embodiments, the oncolytic virus comprises a myxoma virus.

In some embodiments, the immune cells are autologous cells or allogeneic cells.

In some embodiments, the modified immune cells are infected with the oncolytic virus at a multiplicity of infection (MOI) of about 0.1 to an MOI of about 10.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

Viral transduction of cells may be accomplished by any method known in the art. e.g., Palsson, B., et al., (1995), WO95/10619; Morling, F. J. et al., (1995), Gene Therapy, 2:504-508; Gopp et al., (2006), Methods Enzymol, 420:64-81. For example, the infection may be accomplished by spin-infection (also referred to as spin transduction) or “spinoculation” methods that involve subjecting the cells to centrifugation during the period closely following the addition of virus to the cells. In some cases, virus may be concentrated prior to the infection, e.g., by ultracentrifugation.

In some embodiments, the modified immune cells are by spin transduction with the oncolytic virus. In some embodiments, the spin transduction is performed at about 1,000 rpm to about 2,500 rpm (e.g., about 1,000 rpm, 1,100 rpm, 1,200 rpm, 1,300 rpm, 1,400 rpm, 1,500 rpm, 1,600 rpm, 1,700 rpm, 1,800 rpm, 1,900 rpm, 2,000 rpm, 2,100 rpm, 2,200 rpm, 2,300 rpm, 2,400 rpm, 2,500 rpm) for about 1 hour to about 5 hours (e.g., 1, hour, 2 hours, 3 hours, 4 hours, 5 hours).

In some embodiments, the immune cell is infected in the presence of protamine at a concentration ranging from about 5 μg/ml to about 15 μg/ml (e.g., 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml).

b. Compositions and Kits

In another aspect, the above-described immune cells can be incorporated into compositions, e.g., pharmaceutical compositions suitable for administration.

In some embodiments, the composition further comprises non-infected immune cells. In some embodiments, the non-infected immune cells are immune cells that have not been infected with an oncolytic virus, such as myxoma virus. In some embodiments, the non-infected immune cells are the same as the oncolytic virus-infected immune cells, except that they have not been infected with an oncolytic virus, such as myxoma virus.

In some embodiments, the non-infected immune cell is a T cell. In some embodiments, the non-infected immune cell is a tumor-infiltrating T cell or a cytotoxic T lymphocyte. In some embodiments, the non-infected immune cell is the T cell expressing a T cell receptor (TCR) or a chimeric antigen receptor (CAR). In some embodiments, the TCR or the CAR binds specifically to a tumor antigen selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin.

In some embodiments, a ratio of the infected immune cells to the non-infected immune cells is between 1:0.1 and 1:20 (e.g., 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20). In some embodiments, the ratio of the infected immune cells to the non-infected immune cells is about 1:8, about 1:9, about 1:10, or about 1:11. In some embodiments, the ratio of the infected immune cells to the non-infected immune cells is about 1:9.

In some embodiments, the pharmaceutical compositions may include substantially isolated/purified immune cells (oncolytic virus-infected immune cells and optionally non-infect immune cells) and a pharmaceutically acceptable carrier in a form suitable for administration to a subject. Pharmaceutically acceptable carriers are determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

The terms “pharmaceutically acceptable” and “physiologically tolerable,” as referred to compositions, carriers, diluents, and reagents, are used interchangeably and include materials that are capable of administration to or upon a subject without the production of undesirable physiological effects to the degree that would prohibit administration of the composition. For example, “pharmaceutically acceptable excipient” includes an excipient that is useful in preparing a pharmaceutical composition that is generally safe, non-toxic, and desirable, and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, liquid polyethylene glycol, and the like), and suitable mixtures thereof. The proper fluidity can be maintained, e.g., by the use of a coating such as lecithin, by the maintenance of the required particle size in the case of dispersion, and by the use of surfactants.

In some embodiments, the pharmaceutical composition further comprises a therapeutic agent. In some embodiments, the therapeutic agent comprises an anti-tumor or anti-cancer agent.

In some embodiments, the anti-tumor or anti-cancer agent is selected from taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof.

In some embodiments, the therapeutic agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

In some embodiments, the disclosed pharmaceutical compositions can also include adjuvants such as aluminum salts and other mineral adjuvants, tensioactive agents, bacterial derivatives, vehicles, and cytokines. Adjuvants can also have antagonizing immunomodulating properties. For example, adjuvants can stimulate Th1 or Th2 immunity. Compositions and methods as disclosed herein can also include adjuvant therapy.

In some embodiments, the pharmaceutical compositions can be formulated in any conventional manner using one or more physiologically acceptable carriers and/or excipients. The lymphocytes may be formulated for administration by, for example, injection, parenteral, vaginal, rectal administration, or by administration directly to a tumor.

In some embodiments, the pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. In some embodiments, the pharmaceutical compositions can further be formulated as suspensions, solutions or emulsions in oily or aqueous vehicles and may contain other agents, including suspending, stabilizing and/or dispersing agents.

In some embodiments, the pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic to boost immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A carrier can also be a solvent or dispersion medium containing, for example, water, saline, polyol (for example, glycerol, propylene glycol, liquid polyethylene glycol, and the like), suitable mixtures thereof, and vegetable oils. The prevention of the action of microorganisms can be brought about by various antibacterial and antifungal agents known in the art. In many cases, it will be preferable to include isotonic agents, for example, sugars or sodium chloride.

In some embodiments, the above-described immune cells or the composition (e.g., the pharmaceutical composition) can be provided in a kit. In one embodiment, the kit includes a container that contains the immune cells or the composition, and optionally informational material. The informational material can be descriptive, instructional, marketing or other material that relates to the methods described herein and/or the use of the agents for therapeutic benefit. For example, kits may include instruction for the manufacturing, for the therapeutic regimen to be used, and periods of administration. In an embodiment, the kit includes also includes an additional therapeutic agent (e.g., a checkpoint modulator, a chemotherapeutic compound). The kit may comprise one or more containers, each with a different reagent. For example, the kit includes a first container that contains the immune cells or the composition and a second container for the additional therapeutic agent.

In some embodiments, the containers can include a unit dosage of the pharmaceutical composition. In addition to the composition, the kit can include other ingredients, such as a solvent or buffer, an adjuvant, a stabilizer, or a preservative.

In some embodiments, the kit optionally includes a device suitable for administration of the composition, e.g., a syringe or other suitable delivery device. The device can be provided pre-loaded with one or both of the agents or can be empty but suitable for loading.

### Methods of Use

In another aspect, this disclosure also provides a method of treating cancer in a subject. In some embodiments, the method comprises administering to the subject an effective amount of the immune cells or the composition, as described above.

In another aspect, this disclosure further provides a method of treating cancer in a subject. In some embodiments, the method comprises: (a) introducing into a plurality of immune cells a vector comprising a nucleic acid sequence encoding a TCR or CAR to obtain a plurality of modified immune cells; (b) infecting the modified immune cells with an oncolytic virus; (c) optionally culturing the infected immune cells in a cell culture medium; and (d) administering to the subject a composition comprising an effective amount of the infected immune cells.

In some embodiments, the immune cells are capable of inducing autosis of cancer. Autosis is a defined form of cell death induced by excessive autophagy. It is distinct from other forms of cell death, including necrosis and apoptosis, and is characterized by the presence of unique morphological and biochemical features (Jihoon Nah, et al., JACC: Basic to Translational Science, Volume 5, Issue 8, 2020, Pages 857-869). Increasing lines of evidence suggest that cell death through autosis occurs in various cell types in response to some types of stress.

In some embodiments, the method comprises culturing the infected immune cells in a cell culture medium for a period of between 1 and 14 (e.g., 1, 2, 3, 4, 5, 6, 7, 8, 9, 10, 11, 12, 13, 14) days. In some embodiments, the method comprises culturing the infected immune cells in the cell culture medium for a period of 7 days.

In some embodiments, the oncolytic virus comprises a myxoma virus.

In some embodiments, the composition further comprises non-infected immune cells. In some embodiments, a ratio of the infected immune cells to the non-infected immune cells is between 1:0.1 and 1:20 (e.g., 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20). In some embodiments, the ratio of the infected immune cells to the non-infected immune cells is about 1:8, about 1:9, about 1:10, or about 1:11. In some embodiments, the ratio of the infected immune cells to the non-infected immune cells is about 1:9.

In some embodiments, the immune cell is a T cell. In some embodiments, the immune cell is a tumor-infiltrating T cell or a cytotoxic T lymphocyte. In some embodiments, the immune cell is the T cell expressing a T cell receptor (TCR) or a chimeric antigen receptor (CAR). In some embodiments, the TCR or the CAR binds specifically to a tumor antigen selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin.

Generally, adoptive T cell therapy relies on the in vitro expansion of endogenous, cancer-reactive T cells. These T cells can be harvested from cancer patients, manipulated, and then reintroduced into the same or a different patient as a mechanism for generating productive tumor immunity. T cells used in adoptive therapy can be harvested from a variety of sites, including peripheral blood, malignant effusions, resected lymph nodes, and tumor biopsies. Although T cells harvested from the peripheral blood are easier to obtain technically, TILs obtained from biopsies may contain a higher frequency of tumor-reactive cells. Once harvested, T cells can be transfected with a vector as described above.

In some embodiments, a TCR or antigen-binding fragment as disclosed has antigen specificity for an antigen that is characteristic of a disease or disorder. The disease or disorder can be any disease or disorder involving an antigen, such as but not limited to a tumor and/or cancer.

As used herein, the term “subject” may be interchangeably used with the term “patient.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

In some embodiments, the subject is a human. In some embodiments, the subject has cancer. In some embodiments, the subject is immune-depleted.

As used herein, “cancer,” “tumor,” and “malignancy” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods described herein can be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., leukemias and lymphomas) or may comprise solid tumors. The types of cancers to be treated with the disclosed compositions include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

Hematologic cancers are cancers of the blood or bone marrow. Examples of hematologic (or hematogenous) cancers include leukemias, including acute leukemias (such as acute lymphocytic leukemia, acute myelocytic leukemia, acute myelogenous leukemia, promyelocytic, myelomonocytic, monocytic, and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high-grade forms), myeloma Multiple, Waldenstrom's macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

Non-limiting examples of tumors can be treated by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endothelio sarcoma, lymphangiosarcoma, lymphangioendotheliosarcoma, synovioma, testicular cancer, uterine cancer, cervical cancer, gastrointestinal cancer, mesothelioma, cancers associated with viral infection (such as but not limited to human papilloma virus (HPV) associated tumors (e.g., cancer cervix, vagina, vulva, head and neck, anal, and penile carcinomas)), Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma.

Cancers that may be treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lympho epithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglio neuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythro leukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, cancer comprises a solid tumor or a hematologic malignancy. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

The anti-tumor responses after treatment by the methods disclosed herein may be determined in xenograft tumor models. Tumors may be established using any human cancer cell line expressing the TAAs presented by the viral particles. In order to establish xenograft tumor models, about 5×10⁶ viable cells, may be injected, e.g., s.c, into nude athymic mice using, for example, Matrigel (Becton Dickinson). The endpoint of the xenograft tumor models can be determined based on the size of the tumors, weight of animals, survival time, and histochemical and histopathological examination of cancer, using methods known to one skilled in the art.

In some embodiments, the immune cells or the composition, as described herein, may be administered with an additional therapeutic agent or therapy. In some embodiments, the composition can be administered to a subject either simultaneously with, before (e.g., 1-30 days before) or after (e.g., 1-30 days after) the additional therapeutic (including but not limited to small molecules, antibodies, or cellular reagents) that acts to elicit an immune response (e.g., to treat cancer) in the subject. When co-administered with an additional therapeutic, the composition and the additional therapeutic agent may be administered simultaneously or sequentially (in any order). Suitable therapeutically effective dosages for each agent may be lowered due to the additive action or synergy.

In some embodiments, the methods described herein can be combined with additional immunotherapies and therapies. For example, when used for treating cancer, the composition can be used in combination with conventional cancer therapies, e.g., surgery, radiotherapy, chemotherapy or combinations thereof, depending on type of the tumor, patient condition, other health issues, and a variety of factors. In some embodiments, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist, such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments include, for example, aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylenediamine oxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prednisolone); growth factor signal transduction kinase inhibitors; mitochondrial dysfunction inducers and caspase activators; and chromatin disruptors.

In some embodiments, the composition can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can also be combined with other treatments that possess the ability to modulate NKT function or stability, including but not limited to CD Id, CD Id-fusion proteins, CD Id dimers or larger polymers of CD Id, either unloaded or loaded with antigens, CDld-chimeric antigen receptors (CDId-CAR), or any other of the five known CD1 isomers existing in humans (CD la, CD lb, CDlc, CDle), in any of the aforementioned forms or formulations, alone or in combination with each other or other agents.

The pharmaceutical compositions, as described, can be administered in a manner appropriate to the disease to be treated or prevented. In some embodiments, the immune cells or the composition is administered by intravascular, subcutaneous, intraperitoneal, or intratumor injection.

The amount and frequency of administration will be determined by factors such as the condition of the patient, and the type and severity of the patient's disease, although appropriate dosages can be determined by clinical trials.

When “a therapeutically effective amount,” “an immunologically effective amount,” “an effective antitumor quantity,” or “an effective tumor-inhibiting amount” is indicated, the precise amount of the compositions of the present disclosure to be administered can be determined by a physician having account for individual differences in age, weight, tumor size, extent of infection or metastasis, and patient's condition. It can generally be stated that a pharmaceutical composition comprising the lymphocytes described herein can be administered at a dose of 10⁴ to 10⁹ cells/kg body weight, e.g., 10⁵ to 10⁶ cells/kg body weight, including all values integers within these intervals. The lymphocyte compositions can also be administered several times at these dosages. The cells can be administered using infusion techniques that are commonly known in immunotherapy (see, for example, Rosenberg et al., New Eng. J. of Med. 319:1676, 1988). The optimal dose and treatment regimen for a particular patient can be readily determined by one skilled in the art of medicine by monitoring the patient for signs of the disease and adjusting the treatment accordingly.

In some embodiments, the composition can be administered to the subject in a manner compatible with the dosage formulation and in such amount as is therapeutically effective. Dose ranges and frequency of administration can vary depending on, e.g., the nature of the population of immune cells produced by the methods described herein and the medical condition as well as parameters of a specific patient and the route of administration used.

In some embodiments, a population of immune cells produced by the methods described herein can be administered to a subject at a dose ranging from about 10⁷ to about 10¹². A more accurate dose can also depend on the subject in which it is being administered. For example, a lower dose may be required if the subject is juvenile, and a higher dose may be required if the subject is an adult human subject. In some embodiments, a more accurate dose can depend on the weight of the subject.

The administration of the compositions as disclosed can be carried out in any convenient way, including infusion or injection (i.e., intravenous, intrathecal, intramuscular, intraluminal, intratracheal, intraperitoneal, or subcutaneous), transdermal administration, or other methods known in the art. Administration can be once every two weeks, once a week, or more often, but the frequency may be decreased during a maintenance phase of the disease or disorder. In some embodiments, the composition is administered by intravenous infusion.

In some embodiments, the cells, e.g., antigen-specific lymphocytes, are activated and expanded using the methods described herein or other methods known in the art, wherein the cells are expanded to therapeutic levels, before administering to a patient together with (e.g., before, simultaneously or after) any number of relevant treatment modalities.

Also described herein, the compositions can be used in combination with chemotherapy, radiation, immunosuppressive agents, such as cyclosporin, azathioprine, methotrexate, mycophenolate, and FK506, antibodies, or other immunoablating agents such as CAMPATH, anti-cancer antibodies. CD3 or other antibody therapies, cytoxine, fludarabine, cyclosporine, FK506, rapamycin, mycophenolic acid, steroids, FR901228, cytokines, and irradiation.

In some embodiments, the compositions can also be administered to a patient together with (e.g., before, simultaneously or after) bone marrow transplantation, therapy with T lymphocyte ablation using chemotherapy agents such as fludarabine, radiation therapy external beam (XRT), cyclophosphamide, or antibodies such as OKT3 or CAMPATH. Also described herein, the compositions can be administered after ablative therapy of B lymphocytes, such as agents that react with CD20, for example, Rituxan. For example, subjects may undergo standard treatment with high-dose chemotherapy, followed by transplantation of peripheral blood stem cells. In some embodiments, after transplantation, the subjects receive an infusion of the expanded lymphocytes, or the expanded lymphocytes are administered before or after surgery.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the phrases “nucleic acid,” “polynucleotide,” “oligonucleotide,” and “nucleic acid molecule” are used interchangeably to refer to a polymer of DNA and/or RNA, which can be single-stranded, double-stranded, or multi-stranded, synthesized or obtained (e.g., isolated and/or purified) from natural sources, which can contain natural, non-natural, and/or altered nucleotides, and which can contain natural, non-natural, and/or altered internucleotide linkages including, but not limited to phosphoroamidate linkages and/or phosphorothioate linkages instead of the phosphodiester found between the nucleotides of an unmodified oligonucleotide.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule. Or other functional disruption of, or functional addition to, the genetic material of the cell. Exemplary modifications include modifications in the coding region of a heterologous or homologous polypeptide derived from the reference or parent molecule or a functional fragment thereof.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example, a T helper 1 (Th1) or a T helper 2 (Th2) cell. The T-cell can be a helper T-cell (HTL; CD4⁺ T-cell) CD4⁺ T-cell, a cytotoxic T-cell (CTL; CD8⁺ T-cell), a tumor-infiltrating cytotoxic T-cell (TIL; CD8⁺ T-cell), CD4⁺CD8⁺ T-cell, or any other subset of T-cells. Other illustrative populations of T-cells suitable for use in particular embodiments include naive T-cells and memory T-cells. Also included are “NKT cells,” which refer to a specialized population of T-cells that express a semi-invariant ab T-cell receptor, but also express a variety of molecular markers that are typically associated with NK cells, such as NK1.1. NKT cells include NK1.1⁺ and NK1. G, as well as CD4⁺, CD4, CD8⁺, and CD8 cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated a- and b-TCR chains, the TCR in γδ T-cells is made up of a g-chain and a d-chain. γδ T-cells can play a role in immunosurveillance and immunoregulation and were found to be an important source of IL-17 and to induce robust CD8⁺ cytotoxic T-cell response. Also included are “regulatory T-cells” or “Tregs” which refer to T-cells that suppress an abnormal or excessive immune response and play a role in immune tolerance. Tregs cells are typically transcription factor Foxp3-positive CD4⁺ T cells and can also include transcription factor Foxp3− negative regulatory T-cells that are IL-10-producing CD4⁺ T cells.

The terms “natural killer cell” and “NK cell” are used interchangeably and used synonymously herein. As used herein, NK cell refers to a differentiated lymphocyte with a CD 16+CD56+ and/or CD57+TCR− phenotype. NKs are characterized by their ability to bind to and kill cells that fail to express ‘self’ MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician. Thus, the term “treatment” includes preventing a condition from occurring in a patient, particularly when the patient is predisposed to acquiring the condition; reducing and/or inhibiting the condition and/or its development and/or progression; and/or ameliorating and/or reversing the condition. Insofar as some embodiments of the methods of the presently disclosed subject matter are directed to preventing conditions, it is understood that the term “prevent” does not require that the condition be completely thwarted. Rather, as used herein, the term “preventing” refers to the ability of one of ordinary skill in the art to identify a population that is susceptible to the condition, such that administration of the compositions of the presently disclosed subject matter might occur prior to the onset of the condition. The term does not imply that the condition must be completely avoided.

The term “inhibiting cell growth” or “inhibiting proliferation of cells” refers to reducing or halting the growth rate of cells. For example, by inhibiting the growth of tumor cells, the rate of increase in size of the tumor may slow. In other embodiments, the tumor may stay the same size or decrease in size, i.e., regress. In particular embodiments, the rate of cell growth or cell proliferation is inhibited by at least 20%, at least 30%, at least 40%, at least 50%, at least 60%, at least 70%, at least 80%, or at least 90%.

The term “eliciting” or “enhancing” in the context of an immune response refers to triggering or increasing an immune response, such as an increase in the ability of immune cells to target and/or kill cancer cells or to target and/or kill pathogens and pathogen-infected cells (e.g., EBV-positive cancer cells).

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “pharmaceutical composition” refers to a mixture of at least one compound useful within this disclosure with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting an agent within or to the subject such that it may perform its intended function. Typically, such agents are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

“Parenteral” administration of a composition includes, e.g., subcutaneous (s.c.), intravenous (i.v.), intramuscular (i.m.), or intrasternal injection, or infusion techniques.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the subsequent EXAMPLES below.

### Mice

C57BL/6 (B6), DKO-NSG (MHC-I/II-double knockout; NOD.Cg-B2m^{tm1Unc} Prkdc^{scid} H2dl^{Ab1-Ea} Il2rg^{tm1Wj1l/SzJ}), Cd4^{-/-} (B6.129S2-Cd4^{tm1Mak}/J), Cd8^{-/-} (B6.129S2-Cd8a^{tm1Mak}/J), Cd3^{-/-} (B6; 129-Cd3e^{tm1Lov}/J), CD4⁺ TRP-1 (B6.Cg-Rag1^{tm1Mom} Tyrp1^{B-w} Tg(Tcra, Tcrb) 9Rest/J), CD8⁺ TRP-1 (B6.Cg-Rag2^{tm1.1Cgn} Tcrb^{Ln4Sdou} Tcra^{Ln2Sdou}/J) mice were purchased from Jackson Laboratory. Male and female 6- to 8-week-old mice were used for each animal experiment. The studies were approved by the Institutional Animal Care and Use Committee and Institutional Review Board of the Wake Forest School of Medicine.

### Cell Lines

The human OvCa cell line SKOV3; Burkitt's lymphoma cell line Raji; human pancreatic ductal cell line PANC1; human breast cancer cell line SK-BR-3; human multiple myeloma cell line RPMI 8226; murine melanoma cell lines B16; and BSC40 cells, were purchased from ATCC. Human melanoma cell line, Mel-264 (HLA-A2⁺ MART-1⁺) was obtained from Dr. Steven Rosenberg (Hughes, M.S., et al. (2005). Hum. Gene Ther.). Human glioblastoma cell line, U251, was a gift from Dr. Waldemar Debinski. Murine OvCa cell line, ID-8, was a gift from Dr. Neveen Said at Wake Forest School of Medicine. SKP-1-overexpressing SKOV3 (SKOV3-SKP-1) tumor cells were generated by transduction with lentivirus vectors encoding human SKP-1. SKOV3-IFNGR1-KO tumor cells were generated using CRISPR/Cas9 for IFNGR1 deletion. SKOV3-MSLN-KO tumor cells were generated using CRISPR/Cas9 for Msln deletion. B16^{TRP-1-KO} cells were generated using CRISPR/Cas9 for Tyrp1 (TRP-1) deletion. ID-8 tumor cells were KO of Tp53 gene by CRISPR/Cas9 to recapitulate the human high-grade serous OvCa (Walton, J., et al. (2016) Cancer Res. 76, 6118-6129). hMSLN-expressing ID-8 tumor cells were generated by transduction with lentivirus vectors encoding hMSLN to ID-8^{p53-KO} cells. Cells were cultured in RPMI 1640 Medium (Invitrogen) supplemented with 10% heat-inactivated fetal bovine serum (Thermo Scientific), and 100 U/ml penicillin-streptomycin and 2 mM L-glutamine (Invitrogen). Human umbilical vein endothelial cells (HUVECs), human epithelial cells (RWPE-1), and human stromal cells (HS-5) were purchased from ATCC. hMSLN-expressing HUVECs, hMSLN-expressing RWPE-1, and hMSLN-expressing HS-5 were generated by transduction with lentivirus vectors encoding hMSLN.

### Myxoma Virus

Myxoma virus (MYXV) stocks were grown using RK13 or Vero cells purchased from ATCC and purified by centrifugation on a sucrose cushion or sucrose gradient as described previously (Smallwood, et al. (2010). Curr. Protoc. Microbiol.). Construction of Myxv-tdTomato (wild-type MYXV expressing tdTomato under a poxvirus synthetic early/late promoter) and Myxv-Fluc (wild-type MYXV expressing firefly luciferase under a poxvirus synthetic early/late promoter and tdTomato under a poxvirus p11 late promoter) were described previously (Yamada, Y., and Liu, D. X. (2009) J. Virol.; Zemp, F. J., et al. (2013) Neuro. Oncol.).

### Viral Titer Measurement

MYXV titer measurement was performed as described before (Wennier, S. T., et al. (2012) Mol. Ther.). The amount of infectious virus in each sample (culture supernatant or cell lysate) was quantified using foci formation assay on BSC40 cells. In some studies, MSLN CAR-T^{MYXV-tdTomato} or MART-1 T^{MYXV-tdTomato} cells were cocultured with SKOV3 cells, Mel-264 cells, or beads, and culture supernatants (virus containing media) were collected 72 hrs after reactivation. In some studies, treated T^{MYXV-tdTomato} cells were lysed by 2 rounds of frozen and thawed cycles, and cell lysates containing virus were collected. About 48 hrs after adding diluted culture supernatants or cell lysates to BSC40 cells, the numbers of red fluorescent foci were counted from each dilution and calculated for the virus titer.

### Reagents

Human IgG1 isotype control (catalog #BE0297), aTNFα (Clone Infliximab; catalog #SIM0006), aIL-4 (Clone MP4-25D2; catalog #BE0240), aIL-9 (Clone MH9A4; catalog #BE0327), aIL-5 (Clone TRFK5; catalog #BE0198), aIFN-γ (Clone B133.5; catalog #BE0235), and aTGF-β (Clone 1D11.16.8; catalog #BE0057) mAbs were purchased from BioXCell. aIL-3 (Clone 4815; catalog #MAB603), aIL-13 (Clone 32116; catalog #MAB213), aIL-17 (catalog #AF-317-NA), aGM-CSF (Clone 3209; catalog #MAB215), aIL-2 (Clone 5334; catalog #MAB202), human cytokines IFN-γ (catalog #285-IF), and IL-2 (catalog #202-IL) were purchased from R&D Systems. BYK204165 (PARP-1 inhibitor; catalog #B3188), Y-27632 (ROCK inhibitor; catalog #Y0503), lactoferrin (NETs inhibitor; catalog #L9507), and CA-074Me (CTSB inhibitor; catalog #205530) were purchased from MilliporeSigma. PIK-III (VPS34 inhibitor; catalog #103546-876) was purchased from VWR. Tat-Beclin1 D11 (catalog #NBP2-49888) was purchased from Novus Biologicals. siRNAs targeting the human Na⁺, K⁺-ATPase a1 subunit (no. 1: CUCGCUCACUGGUGAAUCA; (SEQ ID NO: 37) no. 2: GAUUCGAAAUGGUGAGAAA; (SEQ ID NO: 38) and no. 3: CAUCCAAGCUGCUACAGAA) (SEQ ID NO: 39) were purchased from Invitrogen (catalog #4390824). Nontargeting siRNA was purchased from Thermo Scientific (catalog #D-001210-02-20). pBJ5/MF-ΔhAkt encoding human constitutively active AKT (CA-AKT) has been constructed as described previously (Sun, J., et al. (2010) Mol. Ther.). Human CD3⁺ T Cell Isolation Kit (catalog #17751), Human CD4⁺ T Cell Isolation Kit (catalog #17952), and Human CD8⁺ T Cell Isolation Kit (catalog #17953) were purchased from STEMCELL Technologies.

### Human T Cell Preparation

Human T cells were activated by priming human CD3+ T cells isolated from PBMCs of the healthy donor with aCD3/CD28 Dynabeads and 200 U/ml hIL-2 for 24 hrs. During the activation, T cells were transduced with hMSLN-(SS1)-hBBZ-CAR (Ho, M., et al. (2011) Int. J. Cancer.) or CD19-(FMC63)-hBBZ-CAR (Sommermeyer, D., et al. (2017) Leukemia) encoding y-retrovirus to make T cells targeting human MSLN or CD19 in the presence of 10 μg/ml protamine sulfate (Sigma) by centrifugation for 2 hrs at 1,800 rpm, at room temperature. T cells may be transduced with CD3Z-truncated hMSLN-hBBAZ-CAR as a control. In some studies, T cells were transduced with human HLA-A*02-restricted MART-1₂₇₋₃₅ specific TCR lentivector (DMF5) to make T cells targeting melanoma-associated antigen MART-1 (Hughes, M. S., et al. (2005) Hum. Gene Ther.) in the presence of 10 μg/ml protamine sulfate (Sigma) by centrifugation for 2 hrs at 1,800 rpm, at room temperature. T cells were then expanded in the presence of 200 U/ml hIL-2 for an additional 7-8 days before use. Transduction rates of CAR or TCR vectors were >85%.

### Murine T Cell Preparation

Murine T cells were activated by priming CD3⁺ T cells isolated from the spleens of C57BL/6 (B6) mice with aCD3/CD28 Dynabeads and 200 U/ml hIL-2 for 24 hrs. During the activation, T cells were transduced with hMSLN-(SS1)-mBBZ-CAR encoding y-retrovirus to make T cells targeting MSLN in the presence of 10 μg/ml protamine sulfate (Sigma) by centrifugation for 2 hrs at 1,800 rpm at room temperature (Ho, M., et al. (2011) Int. J. Cancer.). CAR⁺ (GFP⁺) T cells were sorted and then expanded in the presence of 200 U/ml hIL-2 for an additional 7-10 days before use. In some studies, CD4⁺ TRP-1-specific T cells were prepared by culturing splenocytes CD4⁺ of TRP-1 mice with TRP-1 peptide (SGHNCGTCRPGWRGAACNQKILTVR; 5 μg/ml) (Muranski, P., et al. (2008) Blood 112, 362-373) and hIL-2 (200 U/ml). CD8⁺ TRP-1-specific T cells were prepared by culturing splenocytes of CD8⁺ TRP-1 mice with TRP-1 peptide (TAPDNLGYA; 5 μg/ml) (Dougan, S. K., et al. (2013) Cancer Immunol. Res.) and hIL-2 (200 U/ml). After culturing for a total of 5 days, CD4⁺ TRP-1 T and CD8⁻ TRP-1 T cells were depleted of dead cells and mixed in a ratio of 1:1 for use in animal studies.

### Infecting of MYXV to Tumor-Specific T Cells

To infect MYXV to the tumor-specific T cells, MYXV was added at MOI of 3:1 to T cells 7-8 days after T cell expansion, in the presence of 10 μg/ml protamine sulfate (Sigma) by centrifugation for 2 hrs at 1,800 rpm, at room temperature. Tumor-specific T^{10% MYXV} cells were formulated with tumor-specific T and tumor-specific T^{MYXV} at a ratio of 9:1. % of live tumor-specific T^{MYXV} cells were determined by immunofluorescence. The relative count of viable MSLN CAR-T cells was determined by trypan blue exclusion test.

To test the infectious activity of MYXV, MYXV-tdTomato (3×10⁴ FFU), CD19 CAR-T^{MYXV-tdTomato} (1×10²), or MSLN CAR-T^{MYXV-Tdtomato} (1×10²) was added to Raji or SKOV3 cells (1×10⁴). Relative tdTomato fluorescence intensities were determined with an Eclipse TE300 Inverted Microscope.

### Tumor Models and Treatments

DKO-NSG mice received s.c. injections of SKOV3 cells (1×10⁷), SKOV3-MSLN-KO cells (1×10⁷), SKOV3-SKP-1 cells (1×10⁷), or SKOV3-IFNGR1-KO cells (1×10⁷). MSLN CAR-T cells (2.5×10⁶) or MSLN CAR-T^{10% MYXV} (2.5×10⁶; contains 90% of MSLN CAR-T cells and 10% of MSLN CAR-T^{MYXV} cells) cells were i.v. injected when tumors reached ~9×8 mm (or ~8×7 mm) on day 40. In some experiments, PBS or MYXV was i.t. injected only into tumors on the left flanks (L. i.t.); tumors on the right flanks did not receive i.t. injections. MART-1⁺ Mel-264 cells (2×10⁶) were s.c. injected into DKO-NSG mice. MART-1 T cells (5×10⁶) or MART-1 T^{10% MYXV} cells (5×10⁶; contains 90% of MART-1 T cells and 10% of MART-1 T^{MYXV} cells) were i.v. injected when tumors reached ~7×6 mm on day 10. In some experiments, PBS or MYXV was i.t. injected only into the tumors on the left flanks (L. i.t.), and tumors on the right flanks did not receive i.t. injections. Raji tumor cells (1×10⁵) were i.v. injected into DKO-NSG mice. CD19 CAR-T cells (2.5×10⁶) or CD19 CAR-T^{10% MYXV} cells (2.5×10⁶; contains 90% of CD19 CAR-T cells and 10% of CD19 CAR-T^{MYXV} cells) were i.v. injected on day 10. In some experiments, PBS or MYXV was i.v. injected. Mice were euthanized at indicated days or the endpoint.

B6 mice were received s.c. injection with ID-8^{80% hMSLN} tumors (containing 20% WT ID-8 cells as ALVs). At 43 days (tumors reached ~9×7 mm) after tumor injection, mice were treated with i.v. injection with MSLN CAR-T (5×10⁶) or MSLN CAR-T^{10% MYXV} (5×10⁶) cells. In some studies, B6 mice were inoculated s.c. with 1×10⁶ B16^{20% TRP-1-KO} cells (left flank, containing 20% B16^{TRP-1-KO} AL Vs; no injection on the right flank) and injected i.v. with 1×10⁵ B16^{20% TRP-1-KO} cells to induce lung metastatic tumors. TRP-1 T (5×10⁶) or TRP-1 T^{10% MYXV} (5×10⁶) cells were i.v. injected on day 10 when s.c. tumors reached ~7×6 mm. In some studies, PBS or MYXV was i.t. injected only into the tumors on the left flanks (L. i.t.), and tumors on the right flanks did not receive intratumorally injection. In some studies, WT, CD4^{-/-}, CD8^{-/-} and CD3^{-/-} B6 mice were received s.c. B16^{20% TRP-1-KO} tumors (1×10⁶ B16^{20% TRP-1-KO} challenged s.c. 8 days before ACT). TRP-1 T^{10% MYXV} (5×10⁶) cells were transferred i.v. into the mice when the tumor reached ~7×6 mm. In some studies, WT and CD3^{-/-} B6 mice were s.c. inoculated with B16^{20% TRP-1-KO+Ctrl} (1×10⁶; 80% B16+20% B16^{TRP-1-KO+Ctrl}), B16^{20% TRP-1-KO+SKP-1} (1×10⁶; 80% B16+20% B16^{TRP-1-KO+SKP-1}), or B16 (1×10⁶) tumor cells. TRP-1 T^{10% MYXV} (5×10⁶) cells were i.v. transferred into mice when tumors reached ~7×6 mm (1×10⁶ tumor cells challenged s.c. 8 days before ACT). Cyclophosphamide was given i.p. as a single dose at 120 mg/kg 1 day before T-cell transfer. Mice were euthanized at indicated days or the endpoint.

### (i) In Vivo Bioluminescence Imaging

Before imaging, mice were anesthetized with isoflurane and i.p. injected with 100 ml of 20 mg/ml D-Luciferin (Xenogen Corp.). After 3 min, animals were imaged using an IVIS 200 system (Xenogen), according to the manufacturer's instructions. Living Image software (Xenogen) was used to analyze data.

(ii) In Vitro Cytotoxic Assays The functionality of tumor-specific T^{10% MYXV} cells was assessed by coculturing with target cells in 96-well plates, e.g., SKOV3-Luc⁺, Mel-264-Luc⁺, and Raji-Luc⁺ tumor cells. Unless otherwise stated, 2×10³ sorted TCR/CAR-T cells or 2×10³ sorted TCR/CAR-T^{10% MYXV} cells were mixed with 2×10⁴ target cells in a total volume of 200 μl complete medium. After coculture for 8 to 120 hrs, 1 ml of 20 mg/ml D-Luciferin (Xenogen Corp.) was added to each well, and luminescent signals were analyzed using POLARstar Omega Plate Reader (BMG LABTECH), according to the manufacturer's instructions. Luminescent signals were used to calculate cytotoxicity.

In some studies, human monocytes were isolated from peripheral blood mononuclear cells of the healthy donor using EasySep™ Direct Human Monocyte Isolation Kit (STEMCELL; catalog #19669). hMSLN-expressing monocytes were generated by transduction with expression plasmid encoding hMSLN.

In some studies, SKOV3-Luc⁺ tumor cells were seeded into the upper (2×10⁵) and lower (2×10⁴) Transwell chamber with 400 μl of the completed medium. MSLN CAR-T (2×10⁴) or MSLN CAR-T^{10% MYXV} (2×10⁴) cells were added to the upper Transwell chamber. The pore size of Transwell inserts is 0.4 mM. After 72 hrs of coculture, luminescent signals of SKOV3-Luc+ cells in the lower chamber were detected using POLARstar Omega Plate Reader (BMG LABTECH), according to the manufacturer's instructions.

### RNA-Sequencing

SKOV3 cells were treated with MYXV, MSLN CAR-T cells, MSLN CAR-T^{10% MYXV} cells, or Tat-Beclin1 for 24 hrs in vitro. Total RNA was extracted with the RNeasy Mini kit (Qiagen). In some studies, B16^{20% TRP-1-KO} cells (1×10⁶) were s.c. injected in both flanks of the mice. TRP-1 T (5×10⁶) or TRP-1 T^{10% MYXV} (5×10⁶) cells were administrated on day 7 when tumors reached ~7×6 mm. MYXV was i.t. injected only into tumors on the left flanks and tumors on the right flanks did not receive i.t. injections. Adjuvant cyclophosphamide (i.p.) was administered to mice one day before ACT. Tumor tissues (~100 mg/mice, 12 days after ACT) were harvested and total RNA was extracted with the RNeasy Mini kit (Qiagen). RNA was transferred to cDNA followed by ligation of adapters, and the cDNA was sequenced. RNA-seq analyses were performed by BGI Genomics Co., Ltd.

### Gene Set Enrichment Analyses

Gene set enrichment analyses (GSEA) of the gene expression profiles were implemented using GSEA software (gsea-v3.0, http://software.broadinstitute.org/gsea/downinfects.jsp). P-values were calculated with the Kolmogorov-Smirnov test (threshold=0.01). The false discovery rate (FDR), q value, is the estimated probability that a gene set with a given NES represents a false-positive finding. The threshold for q value in GSEA is 0.25. Gene sets for the mature effector were derived from a publicly available study of the genes differentially expressed by >2 fold in quaternary versus primary cells (Gu, Z., et al. (2016) Bioinformatics 32, 2847-2849).

### Flow Cytometry and Western Blot Analyses

FITC- or PE-conjugated mAbs (1:100 dilution) were used for staining and analyzed using a FACS Fortessa flow cytometer.

For Western blot analysis, β-Actin (8H10D10) (catalog #3700T) mouse mAb and Cleaved Caspase-3 Ab (catalog #4926), Cleaved Caspase-1 (catalog #89332), RIP (E8S7U) XP® (RIPK1, catalog #73271), CD71 (D7G9X) XP® (TFRC, catalog #13113), Phospho-Akt (Ser473) (D9E) XP® (catalog #4060), and Akt (pan) (11E7) (catalog #4685) rabbit mAbs from Cell Signaling Technology were used at a 1:1000 dilution. Anti-PI 3-kinase p100 (F-11) (catalog #sc-365404) and anti-alpha 1 sodium-potassium ATPase/ATP1A1 (F-2) (catalog #sc-514614) mouse mAbs from Santa Cruz Biotechnology were used at a 1:500 dilution. In some experiments, SKOV3 tumor cells were seeded into upper (2×10⁵) and lower (2×10⁴) Transwell chambers with 400 μl of the completed medium. MSLN CAR-T (2×10⁴) or MSLN CAR-T^{10% MYXV} (2×10⁴) cells were added to the upper Transwell chamber. The pore size of Transwell inserts is 0.4 mM. After 24 hrs of coculture, the SKOV3 cells in the upper (after T cell removal) and lower chambers were lysed and used for Western blot analyses.

### Real-Time PCR

Total RNA was extracted from murine tumors and tumor cell lines using the RNeasy Mini kit (Qiagen) according to the manufacturer's instructions. Genes were expressed with specific primers and analyzed using SYBR green real-time PCR (Applied Biosystems). Expression was normalized to the expression of the housekeeping gene GAPDH.

TABLE 1
Primer Sequences

| | SEQ | | SEQ |
|---|---|---|---|
| | ID | | ID |
| | NO | | NO |
| mGAPDH F: 5′- | 1 | mGAPDH R: 5′- | 2 |
| TTGATGGCAACAATCTCCAC-3′ | | CGTCCCGTAGACAAAATGGT-3′; | |
| mTyrp1 F: 5′- | 3 | mTyrp1 R: 5′- | 4 |
| CCCCTAGCCTATATCTCCCTTTT-3′ | | TACCATCGTGGGGATAATGGC- | |
| | | 3′ | |
| mCd3e F: 5′- | 5 | mCd3e R: 5′- | 6 |
| TCAGCCTCCTAGCTGTTGG-3′ | | GTCAACTCTACACTGGTTCCTG- | |
| | | 3′ | |
| mCd8a F: 5′- | 7 | mCd8a R: 5′- | 8 |
| CCGTTGACCCGCTTTCTGT-3′ | | TTCGGCGTCCATTTTCTTTGG-3′ | |
| mCd4 F: 5′- | 9 | mCd4 R: 5′- | 10 |
| CTAGCTGTCACTCAAGGGAAGA-3′ | | CGAAGGCGAACCTCCTCTAA-3′ | |
| mCcr7 F: 5′- | 11 | mCcr7 R: 5′- | 12 |
| CAGGTGTGCTTCTGCCAAGAT-3′ | | GGTAGGTATCCGTCATGGTCT-3′ | |
| mIl12b F: 5′- | 13 | mIl12b R: 5′- | 14 |
| GTCCTCAGAAGCTAACCATCTCC-3′ | | CCAGAGCCTATGACTCCATGTC- | |
| | | 3′ | |
| mSlc11a1 F: 5′- | 15 | mSlc11a1 R: 5′- | 16 |
| GCAGGCCCAGTTATGGCTC-3′ | | CAGGCTGAATGTACCCTGGTC- | |
| | | 3′ | |
| mLef1 F: 5′- | 17 | mLef1 R: 5′- | 18 |
| GCCACCGATGAGATGATCCC-3′ | | TTGATGTCGGCTAAGTCGCC-3′ | |
| hAtp1a1 F: 5′- | 19 | hAtp1a1 R: 5′- | 20 |
| ACAGACTTGAGCCGGGGATTA-3′ | | TCCATTCAGGAGTAGTGGGAG- | |
| | | 3′ | |
| hSell F: 5′- | 21 | hSell R: 5′- | 22 |
| ACCCAGAGGGACTTATGGAAC-3′ | | GCAGAATCTTCTAGCCCTTTGC- | |
| | | 3′ | |
| hIl7r F: 5′- | 23 | hIl7r R: 5′- | 24 |
| CCCTCGTGGAGGTAAAGTGC-3′ | | CCTTCCCGATAGACGACACTC- | |
| | | 3′ | |
| hTcf7 F: 5′- | 25 | hTcf7 R: 5′- | 26 |
| CTGGCTTCTACTCCCTGACCT-3′ | | ACCAGAACCTAGCATCAAGGA- | |
| | | 3′ | |
| hGzmb F: 5′- | 27 | hGzmb R: 5′- | 28 |
| CCCTGGGAAAACACTCACACA-3′ | | GCACAACTCAATGGTACTGTCG- | |
| | | 3′ | |
| hLag3 F: 5′ - | 29 | hLag3 R: 5′- | 30 |
| GCGGGGACTTCTCGCTATG-3′ | | GGCTCTGAGAGATCCTGGGG-3′ | |
| hCtla4 F: 5′- | 31 | hCtla4 R: 5′- | 32 |
| GCCCTGCACTCTCCTGTTTTT-3′ | | GGTTGCCGCACAGACTTCA-3′ | |
| hPdcd1 F: 5′ - | 33 | hPdcd1 R: 5′- | 34 |
| CCAGGATGGTTCTTAGACTCCC-3′ | | TTTAGCACGAAGCTCTCCGAT-3′ | |
| hGAPDH F: 5′- | 35 | hGAPDH R: 5′- | 36 |
| AGTCAACGGATTTGGTCGTATTGGG- | | ACGTACTCAGCGCCAGCATCG- | |
| 3′ | | 3′ | |
| mCxcl10 F: 5′- | 40 | mCd4 R: 5′- | 10 |
| CCAAGTGCTGCCGTCATTTTC-3′ | | CGAAGGCGAACCTCCTCTAA-3′ | |
| mIl12a F: 5′- | 41 | mCcr7 R: 5′- | 12 |
| CAATCACGCTACCTCCTCTTTT-3′ | | GGTAGGTATCCGTCATGGTCT-3′ | |
| mGzmb F: 5′- | 42 | mIl12b R: 5′- | 14 |
| TCTCGACCCTACATGGCCTTA-3′ | | CCAGAGCCTATGACTCCATGTC- | |
| | | 3′ | |
| mIfng F: 5′- | 43 | mSlc11a1 R: 5′- | 16 |
| GCCACGGCACAGTCATTGA-3′ | | CAGGCTGAATGTACCCTGGTC- | |
| | | 3′ | |
| hMSLN F: 5′- | 44 | mLef1 R: 5′- | 18 |
| CCAACCCACCTAACATTTCCAG-3′ | | TTGATGTCGGCTAAGTCGCC-3′ | |
| hPrf1 F: 5′- | 45 | hGzmb R: 5′- | 28 |
| GGCTGGACGTGACTCCTAAG-3′ | | GCACAACTCAATGGTACTGTCG- | |
| | | 3′ | |

### Immunofluorescence

Cells were fixed with 4% PFA for 15 min at room temperature and blocked with blocking buffer (1×PBS/5% normal serum/0.3% Triton™ X-100) for 1 hr, followed by incubation with LC3A/B rabbit pAb with 1:200 (Cell Signaling Technology, catalog #4108) overnight at 4° C. The cells were then incubated with goat anti-rabbit IgG Alexa Fluor® 488 Conjugate (Cell Signaling Technology, catalog #4412). DAPI, which fluoresces blue (Invitrogen, catalog #D1306) was used for nuclear counterstaining. Fluorescence microscopy assessments were performed on an Eclipse TE300 Inverted Microscope (Nikon Microscopy).

### Enzyme-Linked Immunosorbent Spot (ELISpot) Assays

B16^{20% TRP-1-KO} cells (1×10⁶) were s.c. injected into both flanks of B6 mice. TRP-1 T cells (5×10⁶) or TRP-1 T^{10% MYXV} cells (5×10⁶) were i.v. injected on day 7 when tumors reached ~7×6 mm. In some mice, free MYXV was i.t. injected only into the tumors on the left flanks. Mice were sacrificed on ~20 days after tumor inoculation and tumor tissues were minced and digested using a tumor dissociation kit (Miltenyi Biotec). Each host immune cell subset in about 200 mg tumor tissues was isolated by a bead positive selection kit (CD8⁺ or CD4⁺). Isolated cells per 200 mg of tumor tissues were cocultured with irradiated B16^{TRP-1-KO} tumor cells on IFN-γ ELISpot Kit plates (Mouse IFN-gamma ELISpot Kit, R&D Systems) for 48 hrs following the manufacturer's instructions. The plates were imaged and evaluated by a Cellular Technology Limited ELISPOT Analyzer.

### Imaging Mass Cytometry (IMC) Analysis

IMC was performed on the Hyperion™ Imaging System using Maxpar metal-tagged antibodies. In brief, mouse tissue samples were fixed in 4% formalin, and 6 μm thick sections were made and placed on slides. Each antibody was used at manufacturer-recommended concentrations. The IMC data were bead-normalized and de-barcoded by mass cytometry, and visualized by MCD viewer. Imaging data were analyzed by customized software using Python 3.7 (www.python.org). An imaging processing package, opencv-python (https://pypi.org/project/opencv-python/), was used to quantify total areas in red and blue; these were then used to calculate relative antigen positivity.

### Statistical Analyses

For statistical analysis, Student's t-test or ANOVA was used. A P value less than 0.05 was considered statistically significant. Results are presented as mean±s.d. unless otherwise indicated.

### Example 2

### Efficient Delivery of MYXV by Tumor-Specific T Cells

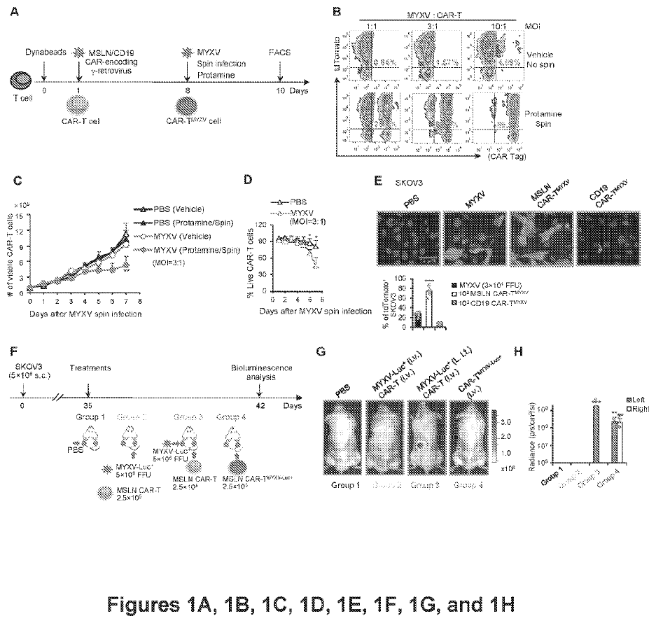

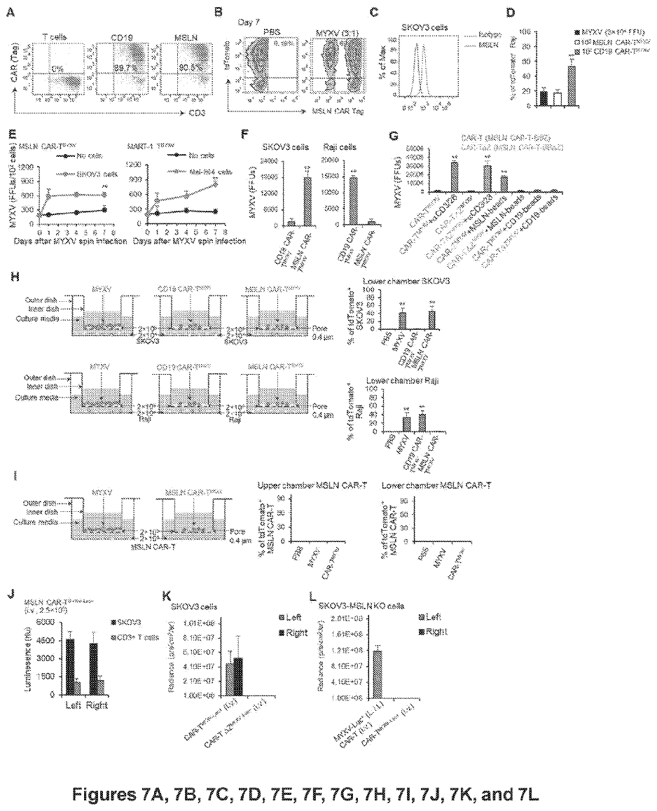

Although MYXV is a promising and safe oncolytic virus that can selectively infect and kill various tumor cells, it is unclear whether non-tumor cells, such as tumor-specific CAR-T cells, can be infected with MYXV. Human mesothelin (MSLN) or CD19 CAR-T cells containing ~90% CAR⁺ cells were prepared (FIGS. 1A and 7A), and expanded for 8 days. As seen in FIG. 1B (top panel, Vehicle/No spin), directly adding tdTomato-expressing reporter MYXV at a multiplicity of infection (MOI) of 1:1, 3:1, 10:1 to CAR-T cells 8 days after CAR-T cell expansion, without spin infection, did not cause significant MYXV infection of T cells, even though T cells were exposed to MYXV for 48 hrs. To infect T cells with MYXV, a related virus transduction protocol was next adopted that is similar to the transduction of CAR-encoding γ-retrovirus to T cells in CAR-T cell preparation, which includes adding protamine and centrifuging for 2 hrs. Interestingly, the MYXV infection rate of CAR-T cells was bolstered by this alternative method to ~80% (FIG. 1B, lower panel, Protamine/Spin). Although adding MYXV to CAR-T cells without spin infection had a limited effect on CAR-T cell survival (FIG. 1C), MYXV-infected CAR-T (CAR-T^{MYXV}, spin-infected) cells had relatively lower proliferation (FIG. 1C) and gradually reduced viability (FIG. 1D) about 7 days after MYXV infection, at which the majority of T cells were still MYXV positive (FIG. 7B). These results indicate that a potential 7-day time frame exists for CAR-T^{MYXV} cells to deliver MYXV into tumor cells.

Next, whether CAR-T^{MYXV} cells can deliver MYXV into tumor cells and whether such a delivery requires CAR-T cell tumor specificity were determined. For CD19⁻MSLN⁺ human SKOV3 ovarian cancer (OvCa) cells (FIG. 7C), adding MSLN CAR-T^{MYXV} but not CD19 CAR-T^{MYXV} cells efficiently delivered MYXV-tdTomato into SKOV3 cells (FIG. 1E). Similarly, CD19 CAR-T^{MYXV} but not MSLN CAR-T^{MYXV} cells delivered MYXV into CD19 MSLN-Raji lymphoma cells (FIG. 7D). Interestingly, MYXV replication in T^{MYXV} cells was detected (FIG. 7E). It was also observed that optimal MYXV release requires CAR activation by antigen-positive tumor cells/beads or anti-CD3/28 bead-mediated T cell activation, and using CD3Z-truncated CAR (CAR-TAZ) did not result in MYXV release (FIGS. 7F and 7G). Notably, adding MYXV-tdTomato to tumor cells (MOI=3) turned fewer tumor cells into tdTomato⁺ cells as compared with adding CAR-T^{MYXV} cells (CAR-T^{MYXV} to tumor cell=1:100) (FIG. 1E), suggesting an efficient virus delivery by CAR-T^{MYXV} cells. In addition, a Transwell assay was used to exclude trogocytosis, which may result in the transfer of tdTomato reporter gene. tdTomato⁺ SKOV3 cells in the lower chamber were observed (FIG. 7H), which may be because MSLN CAR-T^{MYXV} but not CD19 CAR-T^{MYXV} cells were reactivated by SKOV3 cells in the upper chamber and triggered the subsequent release and spread of MYXV to the lower chamber tumor cells. However, adding MYXV or CAR-T^{MYXV} to MSLN CAR-T cells did not cause the infection of MSLN CAR-T cells, indicating that CAR-T^{MYXV} cannot propagate MYXV to other T cells (FIG. 7I). These data highlight an efficient delivery of CAR-T^{MYXV} cells to target cancer cells in an antigen-dependent manner.

To determine whether CAR-T^{MYXV} cell transfer can systemically deliver MYXV in vivo, MSLN CAR-T^{MYXV-luciferase (Luc)+} cells were intravenously (i.v.) injected into MSLN⁺ SKOV3-bearing NSG mice (FIG. 1F). As expected, intratumorally (i.t.) but not i.v. injected MYXV-Luc⁺ is capable of infecting SKOV3 tumors (FIGS. 1G and 1H). Moreover, after injection of MYXV-Luc⁺ into tumors inoculated on one flank of the mice, MYXV did not spread to tumors on the other, non-injected flank (FIGS. 1G and 1H). Remarkably, i.v. transfer of MSLN CAR-T^{MYXV-Luc+} cells efficiently delivered MYXV into SKOV3 tumors inoculated on both flanks of mice, as determined by markedly increased bilateral bioluminescence and the positive bioluminescent signal of isolated tumor cells (FIGS. 1G, 1H, and 7J). This delivery required tumor antigen-mediated CAR signaling because truncation of CD3Z in CAR structure or KO of MSLN in SKOV3 abrogated MYXV delivery (FIGS. 7K and 7L). Overall, the results demonstrate that CAR-T^{MYXV} may be used as efficient carrier cells to systemically deliver MYXV into cognate antigen-expressing tumors.

### MYXV-Infected T Cells Overcome Primary Therapeutic Resistance

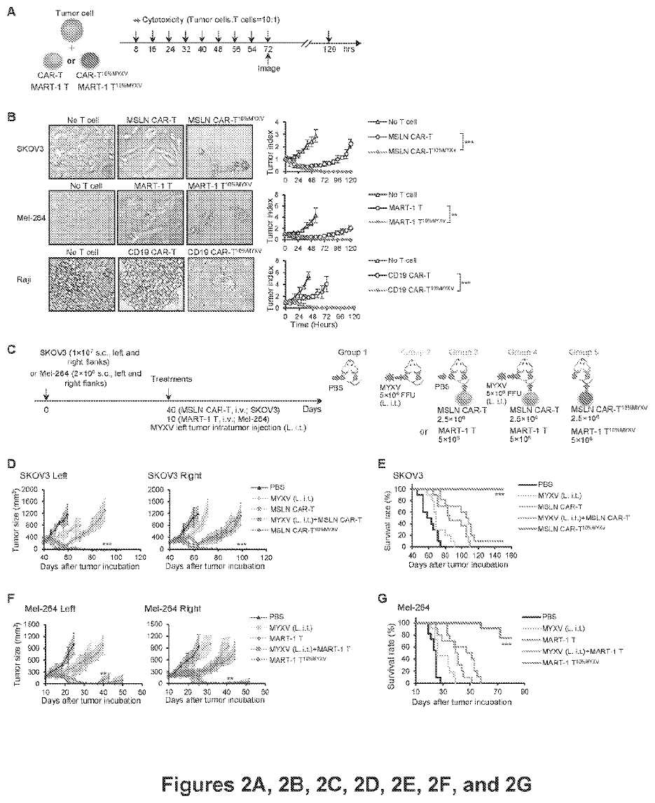

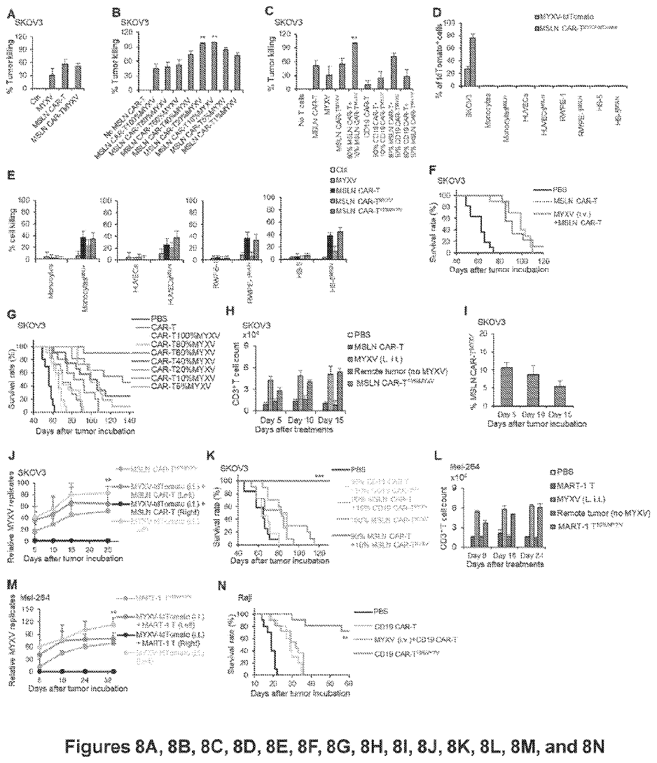

Next, the antitumor potential of MYXV-infected MSLN CAR-T cells was investigated in in vitro cytotoxicity assays. A high ratio of tumor cells to CAR-T cells (10:1) was used, at which CAR-T cells can only lyse a small proportion of tumor cells. Surprisingly, MSLN CAR-T^{MYXV} cells did not display higher antitumor killing ability than uninfected MSLN CAR-T cells (FIG. 8A). Then whether MSLN CAR-T^{MYXV} cells could enhance the baseline antitumor ability of MSLN CAR-T cells was tested. MSLN CAR-T cells were mixed with MSLN CAR-T^{MYXV} cells at various ratios and tested for their function in in vitro cytotoxicity assays. Intriguingly, the optimal tumor-killing activity of CAR-T cells occurred with the formulation of MSLN CAR-T^{10% MYXV} (90% MSLN CAR-T cells+10% MSLN CAR-T^{MYXV} cells) even with a high ratio of tumor cells to CAR-T cells (10:1) (FIG. 8B). Thereafter, T^{10% MYXV} cells were selected to comprehensively evaluate their antitumor function (FIGS. 2A and 2B). Extraordinary tumor-killing activities were also obtained when using CD19 CAR-T^{10% MYXV} cells to target CD19⁺ Raji cells or using MART-1 T^{10% MYXV} cells to target human MART-1⁺ Mel-264 melanoma (FIG. 2B). This synergistic antitumor function seems to require CAR-T^{MYXV} tumor specificity because CD19 CAR-T^{MYXV} cells largely failed to bolster antitumor function of MSLN CAR-T cells in targeting SKOV3 cells (FIG. 8C). However, MSLN CAR-T^{MYXV} cells have a limited capacity to infect non-tumor human monocytes, human umbilical vein endothelial cells (HUVECs), epithelial (RWPE-1) cells, or stromal (HS-5) cells with MYXV, although these cells have been overexpressed MSLN (FIG. 8D). Furthermore, compared with MSLN CAR-T cells, MSLN CAR-T^{10% MYXV} cells did not display increased killing activity against these non-tumor cells (FIG. 8E).

To test whether tumor-specific T^{10% MYXV} cells can also eliminate established tumors in vivo, MSLN CAR-T^{10% MYXV} cells were i.v. injected into MSLN⁺ SKOV3-bearing NSG mice (FIGS. 2C-2E). As a control, MYXV were i.t injected into tumors inoculated on the left flank of mice in combination with i.v. MSLN CAR-T ACT. I.t. injection of MYXV moderately inhibited tumor growth compared with PBS group, followed by aggressive recurrence (FIG. 2D). Combination therapy with i.t injection of MYXV and i.v. MSLN CAR-T ACT largely improved responsiveness of MYXV-injected left tumors, but had no improved effect for right flank tumors without MYXV injection (FIG. 2D), which is in line with the MYXV distribution (FIG. 1H). Similarly, combination therapy with i.v. injection of MYXV and MSLN CAR-T ACT had no improved effect compared with MSLN CAR-T ACT (FIG. 8F). Notably, optimal response was achieved with i.v. MSLN CAR-T^{10% MYXV} cells and resulted in long-term survival (FIGS. 2D, 2E, and 8G), which was associated with significantly increased CAR-T cell infiltration (FIG. 8H), presence of CAR-T^{MYXV} (FIG. 8I), and MYXV replication (FIG. 8J) in the tumor. However, ACT with the formulation of 90% MSLN CAR-T+10% CD19 CAR-T^{MYXV} cells, or 90% CD19 CAR-T+10% CD19 CAR-T^{MYXV} cells, had limited antitumor efficacy compared with MSLN CAR-T^{10% MYXV} cells (FIG. 8K). Robust antitumor efficacy was also observed with MART-1 T^{10% MYXV} ACT for Mel-264 melanoma-bearing NSG mice (FIGS. 2F and 2G), which was accompanied by increased MART-1 T cell infiltration (FIG. 8L) and MYXV replication (FIG. 8M). Finally, CD19 CAR-T^{10% MYXV} ACT displayed enhanced antitumor efficacy in Raji-bearing NSG mice (FIG. 8N). The results thus highlight that antigen specificities of both T and T^{MYXV} cells are crucial for the optimal antitumor efficacy of tumor specific T^{10% MYXV} cells.

### CAR-T^{10% MYXV}-Induced Tumor Cell Autosis Contributes to Tumor Elimination

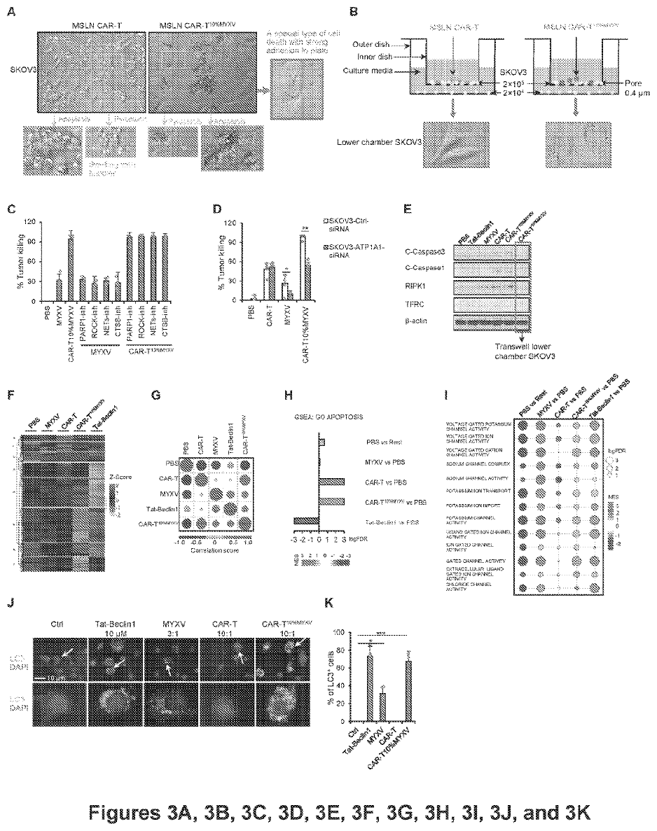

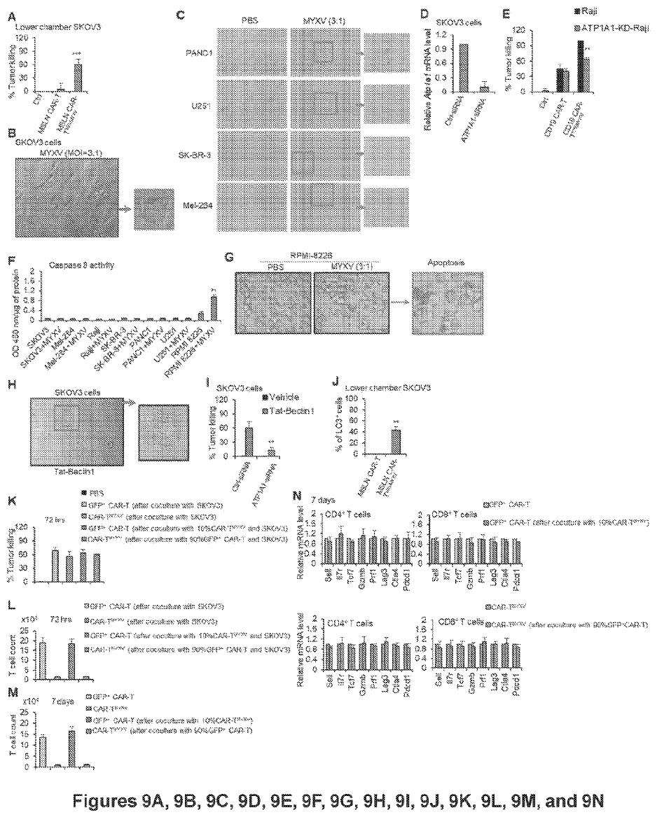

Elimination of tumor cells by T cells depends predominantly on the induction of tumor cell apoptosis and pyroptosis. In an in vitro killing assay, SKOV3 cells mainly underwent apoptosis and pyroptosis when cocultured with MSLN CAR-T cells (FIG. 3A). However, an additional and different type of tumor cell death was observed during the robust killing activity induced by MSLN CAR-T^{10% MYXV} cells, which stands in sharp contrast to what is typically observed with classic apoptosis and pyroptosis (FIG. 3A). This type of tumor cell death has not been attributed to any known T cell-killing pathway before, and appears to be associated with a different morphologic feature that shows firm attachment to culture dishes in vitro (FIG. 3A). Further, this cell death response can also function as bystander killing, since tumor cells separated from the main coculture system by Transwell were not exempt (FIGS. 3B, 9A). This type of tumor cell death was also observed, when treated with MYXV alone, although it seemed to occur at a lower incidence (FIGS. 9B-C). Therefore, inclusion of MYXV-infected T cells in tumor-specific T cells promotes robust tumor clearance, which is associated with an additional type of cell death different from canonical apoptosis or pyroptosis.

To identify the potential death pathway(s) responsible for this unexpected type of cell death, a comprehensive screening of most known types of cell death was carried out (Tang, D., et al. (2019) Cell Res. 29, 347-364). It was found that PARP-1 inhibitor (parthanatos), ROCK inhibitor (entotic cell death), NETs inhibitor (netotic cell death), or CTSB inhibitor (lysosome-dependent cell death) did not inhibit MSLN CAR-T^{10% MYXV} cell-killing activity (FIG. 3C), whereas knockdown (KD) of the Na⁺, K⁺-ATPase α1 subunit (ATP1A1) required for autosis, blocked CAR-T^{10% MYXV} but not CAR-T cell-killing activity (FIGS. 3D, 9D, and 9E). Western blot analysis showed that expression levels of cleaved-caspase3 (apoptosis), cleaved-caspase1 (pyroptosis), RIPK1 (necroptosis), and TFRC (ferroptosis) in tumor cells did not increase after CAR-T^{10% MYXV} cell treatment compared with CAR-T cells, further demonstrating that this special cell death does not belong to apoptosis, pyroptosis, necroptosis, or ferroptosis (FIG. 3E). An exception for this effect is the induction of activated caspase 8-dependent apoptosis in human myeloma cells (FIGS. 9F and 9G). The above analyses strongly suggest that CAR-T^{10% MYXV} cells trigger autosis for tumor clearance.

To further verify that this therapy induced autosis, Tat-Beclin 1 peptide, an established method for autosis induction (Liu, Y., et al. (2013) Proc. Natl. Acad. Sci. U.S.A) was included that can also be blocked by KD of ATP1A1 in tumor cells (FIGS. 9H and 9I). RNA-Seq and cluster analyses were performed, which indicated that CAR-T^{10% MYXV} cells and Tat-Beclin1-treated SKOV3 cells had relatively similar gene profiles (FIG. 3F). In addition, CAR-T^{10% MYXV}-treated tumor cells showed largely overlapping gene signatures with CAR-T cell treatment, including an apoptosis signature (FIGS. 3G and 3H). Interestingly, tumor cells treated with MYXV, CAR-T^{10% MYXV}, and Tat-Beclin1 displayed ~30% shared gene signatures within the top ~200 enriched gene sets, which were distinct from those of cells treated with PBS and CAR-T cells (FIG. 3G). Two prominent cellular changes associated with autosis, ‘ion channel activity’ and ‘ion transport’ signatures (Liu, Y., and Levine, B. (2015) Cell Death Differ.), were also only enriched in MYXV, CAR-T^{10% MYXV}, and Tat-Beclin1 treatments (FIG. 3I). Finally, consistent with the fact that LC3 puncta formation is essential for autosis (Liu, Y., et al. (2013) Proc. Natl. Acad. Sci. U.S.A), largely increased LC3 puncta formation was detected in tumor cells treated with MYXV, CAR-T^{10% MYXV}, and Tat-Beclin1 (FIGS. 3J, 3K, and 9J). Importantly, CAR-T^{MYXV} had limited effects on baseline CAR-T killing activity (FIG. 9K), survival (FIGS. 9L and 9M), T cell features (FIG. 9N), and vice versa (FIGS. 9K-9N). Thus, multiple lines of evidence were assembled to support a potential mechanism underlying CAR-T^{10% MYXV} cell-mediated robust killing activity, representing a mix of classic CAR-T cell-induced apoptosis and pyroptosis with an additional form cell-killing, autosis, to further reinforce antitumor immune responses and tumor clearance.

T Cell-Derived IFN-γ Synergizes with MYXV-Derived M-T5/SKP-1/VPS34 Signaling to Induce Tumor Cell Autosis

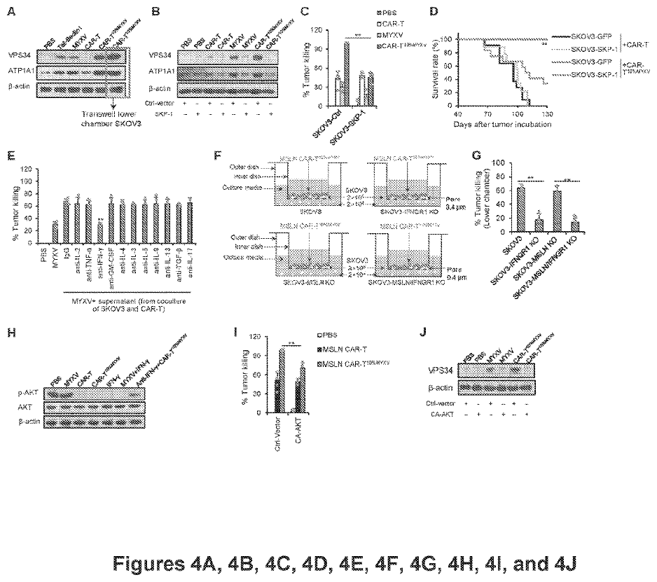

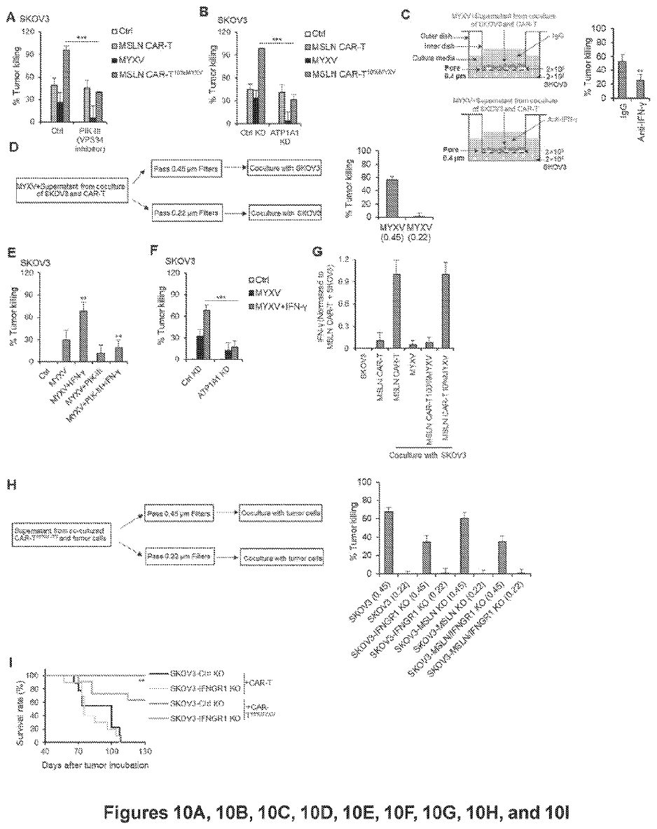

Autosis is a non-apoptotic and non-necrotic form of cell death initiated by excessive accumulation of autophagosomes and due to activation of the Na⁺/K⁺-ATPase pump, changes in membrane osmolarity, and ion transport (Liu, Y., and Levine, B. (2015) Cell Death Differ.). Also different from classic autophagic cell death, cell death in autosis has a distinct morphology and does not require autophagy flux or autolysosomal degradation. To investigate the potential pathway responsible for CAR-T^{10% MYXV} cell promoted-autosis, the MYXV-derived factor(s) were first focused on that may contribute to autophagosome formation. MYXV-encoded M-T5 repeat protein was shown to interact directly with SKP-1 in MYXV-infected cells. Since SKP-1 reportedly forms SCF (SKP-1-CUL1-F-BOX) to ubiquitinate and degrade vacuolar protein-sorting 34 (VPS34), a protein that is crucial for formation of the autophagosome, a series of analyses were performed to uncover the potential role of MYXV-derived M-T5/SKP-1/VPS34 signaling. Indeed, the expression levels of VPS34 and ATP1A1 were substantially increased in SKOV3 cells treated with MYXV or CAR-T^{10% MYXV} (FIG. 4A). Selective inhibition of VPS34 by PIK-III (FIG. 10A) or KD of ATP1A1 (FIG. 10B) reduced the antitumor function of CAR-T^{10% MYXV} but not CAR-T cells. Moreover, overexpression of SKP-1 in SKOV3 cells significantly reduced expression levels of VPS34 and ATP1A1 in SKOV3 cells treated with MYXV or CAR-T^{10% MYXV} (FIG. 4B), and decreased in vitro cytolytic activity of MYXV and CAR-T^{10% MYXV} but not CAR-T cells (FIG. 4C). As a confirmation, tumors established with SKP-1-overexpressing SKOV3 cells were more resistant to CAR-T^{10% MYXV} ACT than SKOV3 cells, but they displayed similar sensitivity to CAR-T ACT (FIG. 4D). These results indicate that tumor cell autosis induced by CAR-T^{10% MYXV} may be triggered, at least in part, by MYXV/M-T5-mediated disruption of SKP-1 to upregulate VSP34 expression/functions.

Because CAR-T^{10% MYXV} cells seem to induce tumor cell autosis more potently than infection with MYXV (FIG. 9B), it was further hypothesized that T cell-derived soluble factor(s) may also contribute to autosis induction. SKOV3 cells were treated with MYXV and supernatant from SKOV3 and CAR-T cell coculture and observed significantly bolstered tumor cell death (FIG. 4E). IFN-γ in the supernatant seems crucial for this effect, as neutralization of IFN-γ but not other cytokines nullified enhanced antitumor activity (FIGS. 4E, 10C, and 10D), and reciprocally, MYXV antitumor function was enhanced by IFN-γ (FIGS. 10E and 10F). However, inhibition of VPS34 by PIK-III (FIG. 10E) or KD of ATP IAI (FIG. 10F) abrogated this effect. These results may also explain the insufficient antitumor activity of 100% MSLN CAR-T^{MYXV} cells because they produced a low amount of IFN-γ when cocultured with tumor cells (FIG. 10G).

Furthermore, IFN-g and MYXV are indispensable for CAR-T^{10% MYXV} cell-mediated bystander killing, since KO of IFNGR1 in SKOV3 cells or removal of MYXV by 0.22-mm filtration largely abrogated tumor eradication in an in vitro killing assay and in tumor-bearing mice (FIGS. 4F-G, 10H-I). Because the mTOR/AKT pathway suppresses autophagosome formation, but can be inhibited by IFN-g, whether AKT can be inhibited by CAR-T^{10% MYXV} cells was tested. Indeed, treatment with IFN-g or IFN-g-producing CAR-T or CAR-T10% MYXY cells markedly reduced p-AKT levels in tumor cells (FIG. 4H). Overexpression of constitutively activate AKT (CA-AKT) largely negated the antitumor activity of CAR-T^{10% MYXV} but not CAR-T cells (FIG. 4I). Moreover, the expression levels of VPS34 were substantially reduced in CA-AKT-overexpressing SKOV3 cells treated with CAR-T^{10% MYXV} (FIG. 4J). Taken together, the results highlight a synergy between MYXV and CAR-T cells to induce tumor autosis death by inhibiting the role of SKP-1 and AKT signaling, respectively, while the elimination of tumor cells remains dependent on T cell tumor specificity.

### Murine Tumor-Specific T^{10% MYXV} ACT Overcomes Acquired Resistance in Solid Tumors

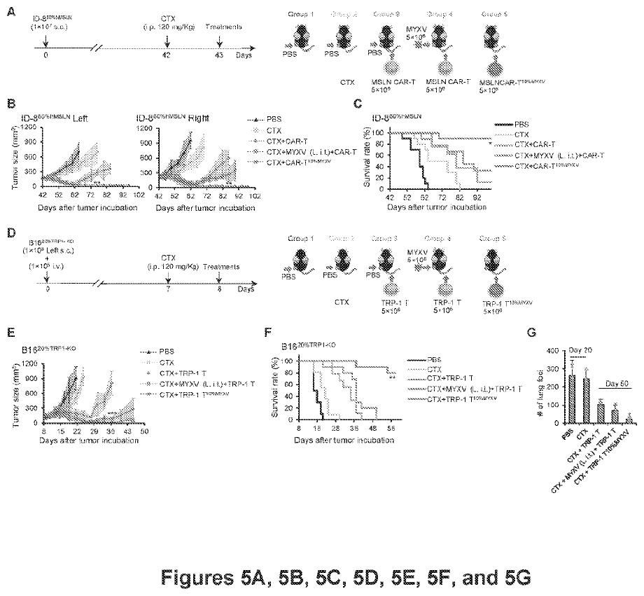

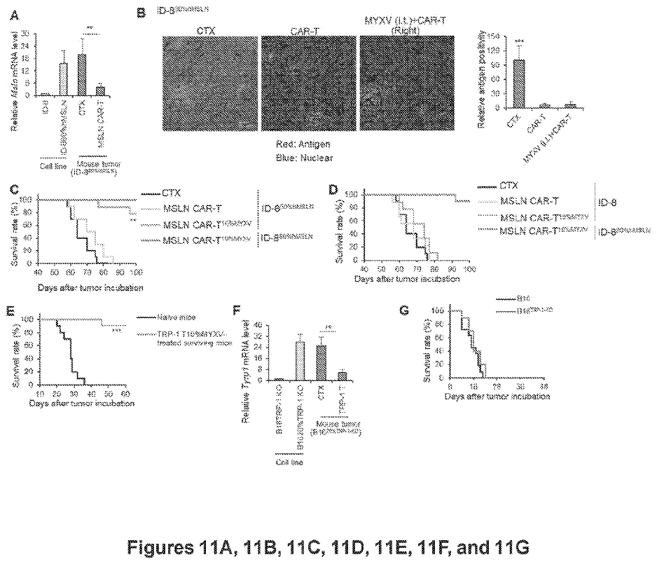

Antigen loss variant (ALV) escape is likely to emerge as a major barrier to durability of CAR-T cell therapy in solid tumors (Majzner, R. G., and Mackall, C. L. (2018) Cancer Discov.). Tumor-specific T^{10% MYXV} ACT may promote bystander killing of AL Vs to overcome this obstacle by inducing host antitumor immune activation and autosis via MYXV delivery into tumor beds. To test this hypothesis, syngeneic tumor models in immunocompetent C57BL/6 (B6) mice were employed. A mixture of 80% antigen-positive and 20% antigen-negative tumor cells was used to establish solid tumors with increased tumor antigen heterogeneity. Murine MSLN CAR-T cells were used to target human MSLN-overexpressed murine ID-8 (ID-8^{hMSLN}) OvCa, and created ID-8^{80% hMSLN} ‘chimeric tumor’ cells containing 20% of MSLN-ID-8 cells serving as ALVs. ACT with murine MSLN CAR-T^{10% MYXV} cells also eliminated tumors inoculated on both flanks of mice and resulted in long-term survival, whereas relapse due to ALV outgrowth seemed inevitable after MSLN CAR-T ACT (FIGS. 5A-5C, 11A-B). However, such antitumor efficacy was slightly reduced when targeting tumors made of 50% ALVs (FIG. 11C) and no antitumor function was detected if tumors were made of 100% ALVs (FIG. 11D).

To strengthen the findings, a tumor model of TRP-1 T cells targeting murine B16 melanoma established with subcutaneous (s.c.) and i.v. injection of B16^{20% TRP-1-KO} tumor cells (80% of B16+20% TRP-1 KO B16 tumor cells) were also used (FIG. 5D). TRP-1 T^{10% MYXV} treatment was highly effective against both s.c. (FIGS. 5E and 5F) and lung metastatic (FIG. 5G) tumors in mice compared with TRP-1 T cell ACT. Remarkably, TRP-1 T^{10% MYXV} ACT also extended protection to mice upon tumor-specific rechallenge (FIG. 11E). Relapsed tumors following TRP-1 T cell ACT appeared to experience ALV outgrowth, although tumors composed of exclusively B16 or TRP-1-KO B16 cells had similar growth rates in vivo (FIGS. 11F-G). Collectively, the results pinpoint a pivotal role of CAR/TCR-T^{10% MYXV} ACT that allows the clearance of solid tumors containing ALVs.

T^{10% MYXV} ACT Induces Autosis and Adaptive Immunity that Restrains ALVs

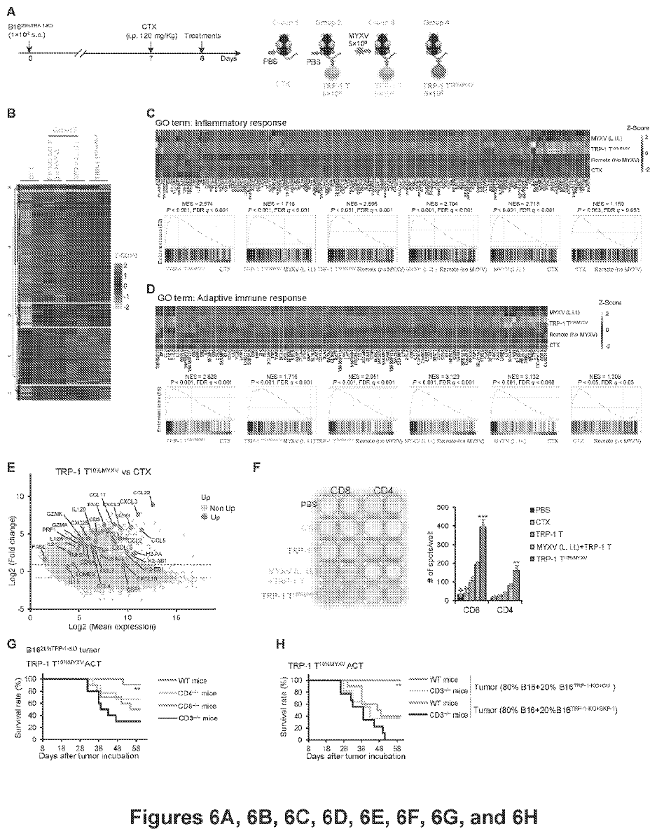

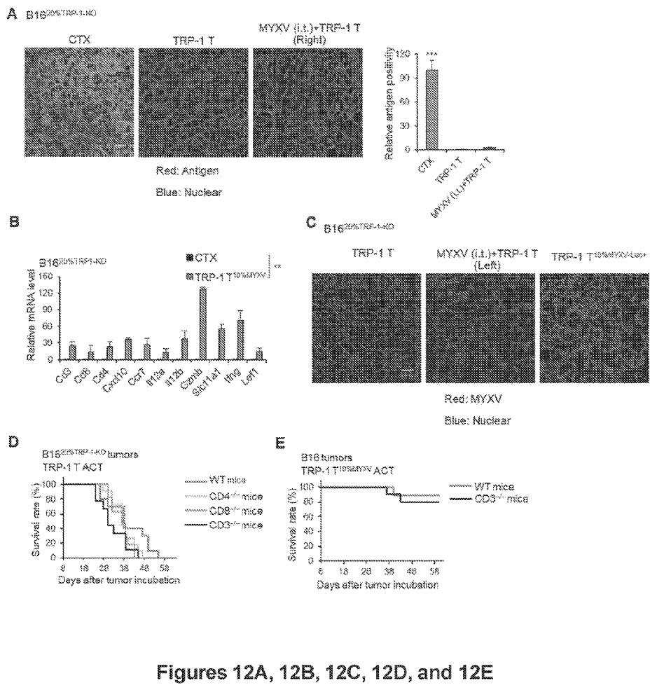

To determine the underlying mechanism within tumor-specific T^{10% MYXV} cells that eliminates solid tumors with antigen heterogeneity (FIG. 12A), global RNA expression of treated B16^{20% TRP-1-KO} tumors was analyzed using RNA-seq (FIG. 6A). Clustering analysis indicated that TRP-1 T^{10% MYXV} ACT resulted in a relatively similar gene profile as MYXV (i.t.) combined with TRP-1 T cell ACT [′MYXV (L. i.t.)′], whereas these two groups were completely different from the ‘remote tumor (no MYXV)’ and control (CTX) treatment (FIG. 6B). Specifically, GSEA revealed that tumors treated with TRP-1 T^{10% MYXV} or ‘MYXV (L. i.t.)’ were highly enriched for ‘inflammatory response’ and ‘adaptive immune response’ signatures (FIGS. 6C and 6D). Mean differential expression analyses further revealed many highly and significantly expressed cytokine and chemokine-related genes and genes suggesting T cell activation in TRP-1 T^{10% MYXV}-treated tumors versus CTX control (FIGS. 6E, 12B).

Given that MYXV can be delivered into tumors, host antitumor immunity was defined by IFN-g ELISpot analysis of tumor-infiltrating CD4⁺ or CD8⁺ host T cells (CD45.1⁺) isolated and restimulated with irradiated B16^{TRP-1-KO} tumor cells ex vivo. The results demonstrated that TRP-1 T^{10% MYXV} ACT induced strong host CD4+ and CD8+ T cell activation (FIG. 6F). Although host T cells do not seem to contribute significantly to the efficacy of TRP-1 T cell ACT targeting B16^{20% TRP-1-KO} tumors (FIG. 12D) or TRP-1 T^{10% MYXV} ACT targeting B16 tumors (FIG. 12E), deficiency of host CD4⁺, CD8⁺, or CD3⁺ T cells significantly reduced the antitumor effect of TRP-1 T^{10% MYXV} ACT targeting B16^{20% TRP-1-KO} tumors (FIG. 6G). Finally, autosis induction in the tumor bed may also contribute to anti-ALV activity of TRP-1 T^{10% MYXV} ACT, because survival was significantly reduced in mice bearing chimeric tumors containing SKP-1-overexpressing autosis-resistant TRP-1-KO B16 tumor cells (FIG. 6H). Taken together, the results demonstrate that both TRP-1 T^{10% MYXV} ACT-promoted host T cell activation and tumor cell autosis may be required to control the clonal expansion of ALVs.

### Discussion

In this study, the potential to exploit CAR-T and TCR-T cells as MYXV-delivery carrier cells was investigated by pre-infecting the T cells with MYXV ex vivo by a spin-infection protocol (CAR-T^{MYXV} and TCR-T^{MYXV}). Tumor-specific CAR-T^{MYXV} and TCR-T^{MYXV} cells efficiently delivered MYXV into the cognate tumor cells, but not normal cells, in an antigen-specific manner. Unexpectedly, a special tumor cell death induced by CAR-T^{MYXV} was observed, which has not been attributed to any T cell killing mechanism before but may contribute to the exciting observed antitumor potency. Differing from classic apoptosis and pyroptosis induced by T cells, this special type of cell death, called autosis, can also mediate a potent bystander killing for antigen-negative tumor cells. Therefore, we performed a series of studies to investigate the functions of these mechanisms.

As described above, a special strategy was developed for systemically delivering oncolytic MYXV into solid tumors by transferring tumor-specific T^{10% MYXV} cells. Remarkably, eradication of solid tumors by tumor specific T^{10% MYXV} cells was associated with induction of tumor cell autosis, which is unlike the classic cytolytic machinery of T cell cytotoxicity and causes a potent bystander killing of tumor cells. Therefore, this example uncovers an unexpected, novel T cell-killing mechanism of tumor cells by T^{10% MYXV}, which could catalyze a paradigm shift in ACT to overcome therapeutic resistance in solid tumors.

MYXV can selectively infect and kill a broad variety of non-rabbit cancerous cells while sparing the normal cell and tissue counterparts. It was found that directly adding MYXV did not efficiently infect 8-day cultured CAR-T cells in vitro. However, successful transduction of MYXV to CAR-T cells ex vivo was achieved by using a modified protamine spin infection protocol similar to CAR-encoding y-retrovirus transfection to T cells. Thus, it was demonstrated that CAR-T cells can be efficiently infected with MYXV under such non-physiological conditions, which turns them into MYXV carriers. Interestingly, CAR-T^{MYXV} cells had only moderate antitumor function, similar to non-infected CAR-T cells. Infection with MYXV may have somehow reduced the effector functions of CAR-T^{MYXV} cells, because decreased IFN-g production was observed when cocultured with tumor cells. It was hypothesized that CAR-T^{MYXV} plus non-infected CAR-T cells might achieve dual-functional effects, maintaining full cytotoxicity of CAR-T cells and augmenting with MYXV delivery into the tumor beds. In fact, a formulation of CAR-T cells containing 10% CAR-T^{MYXV} displayed remarkably potent antitumor functions.

The cytolytic function of T cells mainly induces apoptosis and pyroptosis of cancer cells by recruiting cytolytic machinery to the T cell/cancer cell synapse and the subsequent release of lytic granules containing perforin and granzymes (Liu, Y., et al. (2020) Sci. Immunol.; Jenkins, M. R., and Griffiths, G. M. (2010) Curr. Opin. Immunol.). In this study, induction of tumor cell apoptosis and pyroptosis by a suboptimal dose of CAR-T cell coculture (i.e., tumor cell-to-T cell ratio of 10:1) was insufficient to kill the majority of tumor cells. An intriguing finding is that, even at such a low tumor cell-to-T cell ratio, CAR-T^{10% MYXV} cells appeared to be endowed with unprecedented cytolytic function to eliminate tumor cells. In sharp contrast to apoptosis and pyroptosis, following which solid tumor cells usually detach from the culture dish, a different type of tumor cell death was observed, featuring a unique morphological feature that includes continued firm attachment to culture dishes. This tumor cell death as autosis, and in this scenario, the formation of an immunological cell-cell synapse between tumor and T cells seemed unnecessary for induced tumor cell autosis were further identified. Instead, CAR-T^{MYXV}-released MYXV infection of tumor cells initiates autosis, while optimal levels of tumor cell autosis induction require synergy from CAR-T cell-derived IFN-g. Hence, it is possible that engineering IFN-g-producing MYXV can also enhance MYXV antitumor activity.

Collectively, the results highlight a pivotal role of CAR-T^{10% MYXV} cells in improving the efficacy of ACT by: (1) delivering MYXV into tumor beds; (2) recognizing and inducing classic tumor cell apoptosis and pyroptosis to antigen-positive tumor cells accompanied by IFN-g secretion; (3) inducing autosis in antigen-positive and -negative cancer cells; and (4) potentially eliminating ALVs by autosis and adaptive antitumor immunity. Thus far, the data suggest the existence of a novel tumor cell autosis-triggering strategy dependent on both MYXV and antigen-programmed CAR-T cells, which strategically incorporates MYXV and tumor-specific T cells to overcome therapeutic resistance in solid tumors.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A mammalian immune cell infected with an oncolytic virus.

2. The immune cell of claim 1, wherein the oncolytic virus comprises a myxoma virus.

3. The immune cell of claim 1, wherein the oncolytic virus comprises a reporter gene.

4. The immune cell of claim 1, wherein the immune cell is a lymphocyte.

5. The immune cell of claim 1, wherein the immune cell is a T cell.

6. The immune cell of claim 5, wherein the T cell is a tumor-infiltrating T cell or a cytotoxic T lymphocyte.

7. The immune cell of claim 5, wherein the T cell expresses a T cell receptor (TCR) or a chimeric antigen receptor (CAR).

8. The immune cell of claim 7, wherein the TCR or CAR binds specifically to an antigen on a tumor selected from CD3, CD4, CD5, CD8, CD45, CD19, CD20, CD21, CD22, CD23, CD25, CD40, CD42b, CD69, CD70, CD79, CD80, CD85, CD86, CD137, CD138, CD252, CD268, or mesothelin.

9. The immune cell of claim 5, wherein the tumor comprises a solid tumor.

10. A composition comprising a plurality of immune cells according to claim 1.

11. The composition of claim 10, further comprising non-infected immune cells.

12. The composition of claim 11, wherein the non-infected immune cells are the same as the immune cells infected with the oncolytic virus, except that the non-infected immune cells have not been infected with the oncolytic virus.

13. The composition of claim 11, wherein a ratio of the infected immune cells to the non-infected immune cells is between 1:0.1 and 1:20.

14. The composition of claim 13, wherein the ratio of the infected immune cells to the non-infected immune cells is about 1:9.

15. A kit comprising the immune cell of claim 1.

16. A method of preparing the immune cell according to claim 1, comprising:
(a) introducing into a plurality of immune cells a vector comprising a nucleic acid sequence encoding a TCR or CAR to obtain a plurality of modified immune cells;
(b) infecting the modified immune cells with an oncolytic virus; and
(c) optionally culturing the infected immune cells in a cell culture medium.

17-23. (canceled)

24. A method of treating cancer in a subject, comprising administering to the subject an effective amount of the immune cells of claim 1.

25. (canceled)

26. The method of claim 24, wherein the immune cells are capable of inducing autosis of the cancer.

27-45. (canceled)
